(19) United States
(12) Patent Application Publication
(10) Pub. No.: US 2026/0035436 A1
(43) Pub. Date: Feb. 5, 2026

(54) COMPOSITIONS AND METHODS FOR DIRECTED LIGAND ANTIBODY DEVELOPMENT

(71) Applicant: ABBRATECH INC., Branford, CT (US)

(72) Inventor: Michael P. Weiner, Guilford, CT (US)

(21) Appl. No.: 18/874,292

(22) PCT Filed: Jun. 23, 2023
(86) PCT No.: PCT/US2023/026059

§ 371 Date: Dec. 12, 2024

Related U.S. Application Data
(60) Provisional application No. 63/355,877, filed on Jun. 27, 2022.

(51) Int. Cl. C07K 16/00; C12N 9/10; C12N 9/50; C40B 30/04; C40B 40/10; C40B 50/06

(52) U.S. Cl. CPC C07K 16/005; C12N 9/1044; C12N 9/50; C12Y 203/02013; C12Y 304/22; C40B 30/04; C40B 40/10; C40B 50/06; C07K 2317/10; C07K 2317/55; C07K 2317/565; C07K 2317/622

(57) ABSTRACT

The present disclosure relates to compositions and methods of generating antibodies against a target protein.

### CROSS-REFERENCE TO RELATED APPLICATIONS

The present application claims priority to, and the benefit of, U.S. Provisional Patent Application No. 63/355,877, filed 27 Jun. 2022, which is incorporated herein in its entirety for all purposes.

### FIELD OF THE INVENTION

The present disclosure relates to compositions and methods of generating antibodies against a target protein.

### BACKGROUND

Monoclonal antibody treatment is an immunotherapy that utilizes a monoclonal antibody (mAb) that binds monospecifically to a molecule, such as a protein, of interest. The therapy seeks to stimulate the subject's immune system to target the molecule of interest and/or the cell or organism molecule of interest is present. While it is possible to create monoclonal antibodies that are specific to any target, some targets are more difficult to obtain high affinity antibodies. For example development of antibodies to transmembrane receptors, enzymes or structural proteins by traditional animal immunization and in vitro screening methods is challenging. While select antibodies that target membrane proteins exist, the success rate of development is nonetheless poor compared with antibodies that target soluble or peripherally anchored proteins. Additionally, most of these antibodies do not modulate membrane protein function.

There is a need for improved methods and compositions for the development of affinity reagents that can recognize and modulate membrane proteins. This disclosure fulfills this need.

### SUMMARY

The present disclosure provides methods of generating an antigen binding region (e.g., antibody or an antigen binding fragment thereof) against a target protein, by providing a bifunctional compound comprising an antigen binding region (e.g., antibody or antigen binding fragment thereof, such as scFv) conjugated to a ligand that binds to the target protein; generating a first antibody library by mutating one or more amino acids in the antigen binding regions; screening the first library to identify one or more bifunctional compounds (e.g., one or more bifunctional compounds with an antigen binding region that binds to a target protein) with improved binding affinity to the target protein as compared to the ligand; generating a second library by mutating the ligand (e.g., mutating one or more amino acids of a ligand that is a peptide or protein) of the one or more bifunctional compounds; screening the second library to identify one or more antigen binding regions (e.g., antibodies or antigen binding fragment thereof) that bind to the target protein. In any aspect or embodiment described herein, the ligand is a peptide or small molecule compound. In any aspect or embodiment described herein, the ligand is conjugated to the antigen binding region via a covalent bond, such as a disulfide bond. Alternatively, in any aspect or embodiment described herein, the ligand is conjugated to the antigen binding region via a sortase reaction or a transglutamase reaction. In any aspect or embodiment described herein, the ligand is conjugated in the complementarity-determining region (CDR) of the antigen binding region (e.g., an antibody or antigen binding fragment thereof). In any aspect or embodiment described herein, the antigen binding region includes or is an antibody or an antigen binding fragment thereof (e.g., the antigen binding region is part of an antibody or an antigen binding fragment thereof). In any aspect or embodiment described herein, the ligand is conjugated to the N-terminus of a light chain variable region. In any aspect or embodiment described herein, the ligand is conjugated to the C-terminus of a light chain variable region. In any aspect or embodiment described herein, the ligand is conjugated to the N-terminus of a heavy chain variable region of the antibody. In any aspect or embodiment described herein, the ligand is conjugated to the C-terminus of a heavy chain variable region of the antibody. Alternatively, in any aspect or embodiment described herein, the ligand is conjugated in a framework region of the variable light chain of the antibody. In any aspect or embodiment described herein, the ligand is conjugated in the variable heavy chain of the antibody. In any aspect or embodiment described herein, the antigen binding region includes or is an antigen binding fragment of an antibody (e.g., the antigen binding region is part of an antigen binding fragment of an antibody, such as single chain variable fragments (scFv), a fragment antigen-binding (Fab), or a Fab′).

In any aspect or embodiment described herein, the antigen binding fragment of the antibody is a single chain variable fragment (scFv) comprising a heavy chain variable region, a light chain variable region, and a scFv peptide linker (e.g., 10 to about 25 amino acids, or 10, 11, 12, 13, 14, 15, 16, 17, 18, 19, 20, 21, 22, 23, 24, or 25 amino acids) that links the heavy chain variable region and the light chain variable region, wherein the ligand is conjugated to the scFv peptide linker.

In any aspect or embodiment described herein, the ligand is conjugated to the antigen binding region (e.g., antibody or an antigen binding fragment thereof) via a linker. In any aspect or embodiment described herein, the linker is a peptide linker or protein linker. For example, in an any aspect or embodiment described herein, the linker peptide is about 3 to about 50 amino acids in length. Preferably, in any aspect or embodiment described herein, the linker peptide is about 3 to about 21 amino acids in length.

In any aspect or embodiment described herein, the first library is a phage display library. In any aspect or embodiment described herein, the second library is a phage display library.

Further provided by the present disclosure is a library comprising a plurality of bifunctional compounds, wherein each bifunctional compound comprises an antigen binding region (e.g., a unique antigen binding region) tethered or conjugated to a ligand that binds to a target protein.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present description. These additional advantages objects and embodiments are expressly included within the scope of the present disclosure. The publications and other materials used herein to illuminate the background of the present disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the present disclosure. The drawings are only for the purpose of illustrating specific embodiments of the present disclosure and are not to be construed as limiting the present disclosure. Further objects, features, and advantages of the disclosure will become apparent from the following detailed description taken in conjunction with the accompanying figures showing illustrative embodiments of the disclosure.

FIGS. 1A, 1B, and 1C. (FIG. 1A) Previous design (Directed Ligand Binding (DLB)): Phage displayed single-chain variable fragment (scFv) was incorporated with G Protein-coupled Receptor (GPCR) ligand onto its CDR-H2 or CDR-H3 and these scFv was guided to sit on the active site of the GPCR extracellular region. (FIG. 1B) Directed Ligand Assay (DLA) as described herein: phage displayed scFv are incorporated with GPCR ligand at the peptide linker region (shown as a dotted line) connecting the heavy chain variable domain (VH) and the light chain variable domain (VL). Genetic coding and enzymatic coupling strategies are both indicated. (FIG. 1C). Enzymatic coupling reaction using Sortase, a site-specific peptidyl ligase that recognizes a “LPXTG” site inside the linker region, and then covalently conjugates the ligands with “(G)n” at the N-terminus onto the C-terminus of “LPXT” sequence. Example GPCR CXCR4 is binding its antagonist ligand CVX15 (PDB: 3OE0), membrane are shown. ScFv model is at the top in FIGS. 1A and 1B, from Herceptin Fab structure (PDB: 4 HKZ).

FIG. 2. The schematic workflow of Directed Ligand Assay (DLA) technology. GPCRs are shown. Ligands are shown therein. The scFvs are in ribbon with space-filling at background (PDB: 3AUV]). Linkers are black dotted lines. Final full-length IgG is shown in space-filling (PDB: 1IGT): (A) ligand-receptor pair is identified. Ligands can be either peptides, proteins, or chemical compounds. Genetic-coding or enzyme-mediated coupling is used to tether the ligand onto the VH-VL linker of scFv, which is displayed on a DLA phage display library (B) GPCR over-expressing cell line is used for whole cell panning with the DLA phage library. At first, bio-panning is against a non-relevant GPCR over-expressing cell line. (C) Then the phage library is bio-panned against the target GPCR over-expressing cell line, during which free ligands are used as competitor to increase the stringency. (D) Target-specific binders are washed and selected. (E) Positive binder(s) is (are) eluted and expanded for next screening. (F) Flow cytometry is used for sorting, screening, and validation, based on which bio-panning is repeated 2-3 rounds. (G) The flow validated positive monoclonal binders are converted to full-length IgG for functionality test (e.g., agonist, antagonist, partial agonist, etc.) in a cell-based reporter assay. (H) Final antibodies (Abs) sit on the active site of the GPCR and modify its function.

FIGS. 3A and 3B. The rationale of utilizing the scFv peptide linker in Directed Ligand Assay (DLA). (FIG. 3A) The VH chain and VL chain from the ScFv is individually folded into a characteristic immunoglobulin fold structure composed of 9 strands of closely packed beta-sheets. The large area from VH and VL interaction is buried. VL is on the left side of the right panel and VH is on the right side of the left panel. (FIG. 3B) The Directed Ligand Assay (DLA) takes advantages of the flexible VL-VH linker to incorporate the GPCR ligand using genetic-coding or enzymatic-coupling methods. VL-VH linkers are a doted line. The ScFv model is derived from Herceptin Fab structure (PDB: 4 HKZ]), CDR regions are in ribbon (L1, L2, L3, H1, H2, H3).

FIGS. 4A and 4B. Preliminary data of Sortase conjugated DLB library. (FIG. 4A) Flow cytometry validation of poly-clonal libraries after several bio-panning rounds. Histamine ligand was conjugated in bulk using sortase to the modified CDR-H3 of a high titer DLB hyper phage library. The non-conjugated and conjugated phage libraries were then tested by flow cytometry for a population shift against the histamine receptor OE cell lines (black: no ligand conjugated library, white: conjugated with histamine library). (FIG. 4B) APJ IgG clones in a reporter cell assay. Three single cones are either non-conjugated (IgGs 1, 2, and 3) or conjugated via sortase with Apelin ligand (IgGs 1c, 2c, 3c). They were tested in a reporter cell assay under agonist mode and antagonist mode side-by-side. Even though all six monoclonal antibodies (mAbs) bind to OE cells (data not shown), the preliminary functional results indicate that these APJ IgGs are only functional when conjugated to the ligand.

FIGS. 5A and 5B. Affinity Maturation of DLA antibodies show increased binding affinity and altered functionalities. (FIG. 5A) Flow cytometry shift of affinity matured (AffMatted) CXCR4 variant B9 and Discovery CXCR4 binder A10_1 show that AffMatted CXCR4 binder increases binding affinities to GPCR on cell. (FIG. 5B) GPCR calcium assay shows AffMatted CXCR4 variant binder B9 changed its functionality from antagonist to partial agonist.

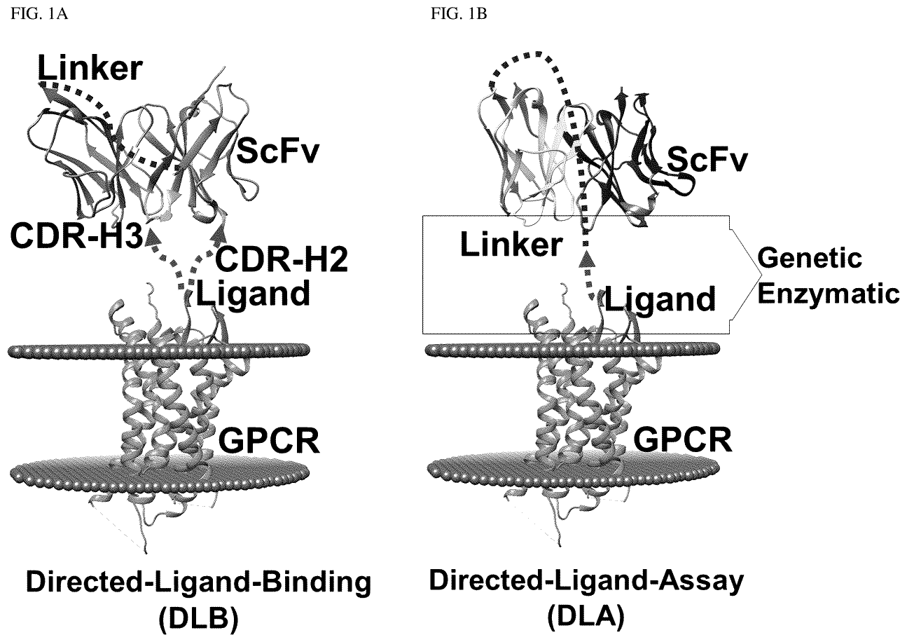

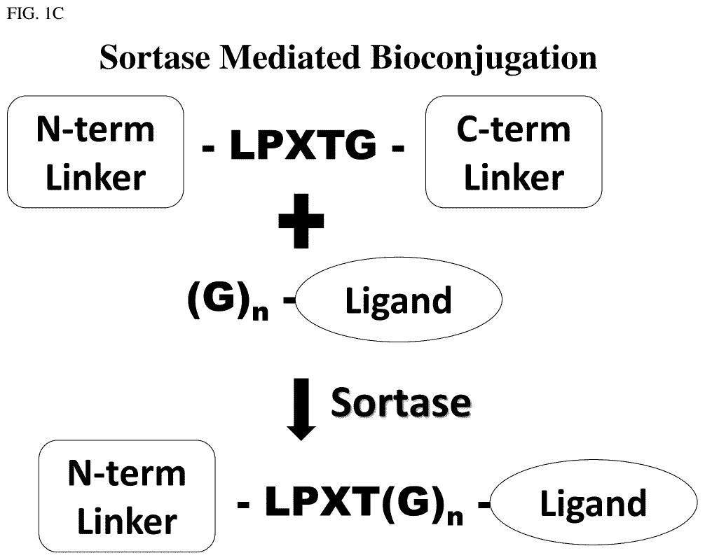

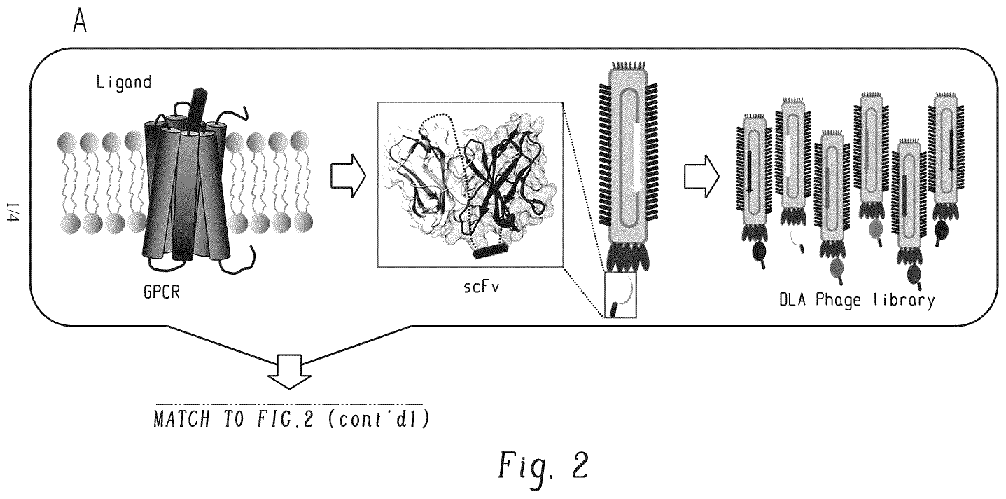

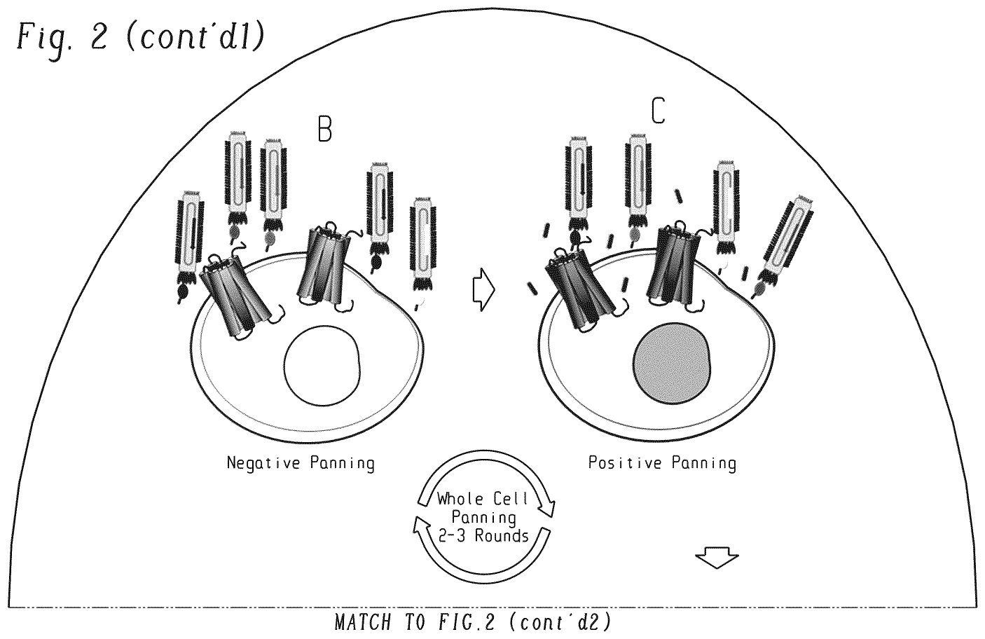

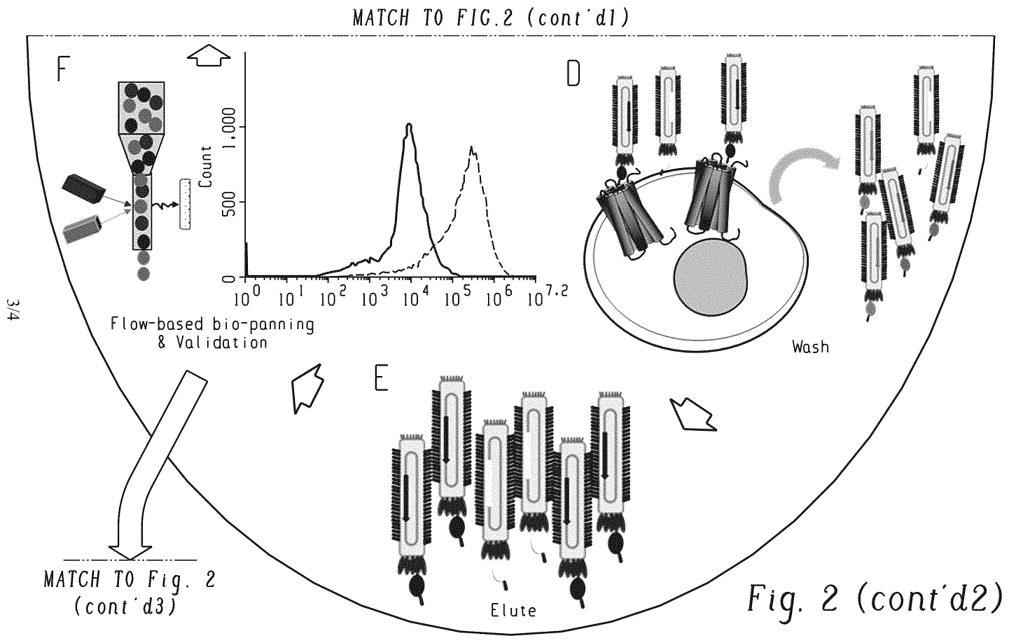

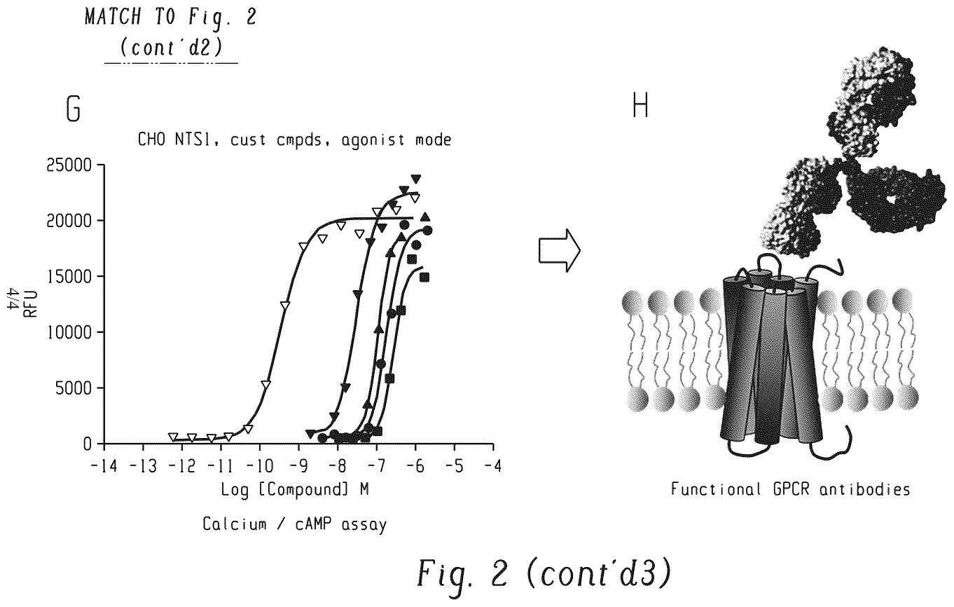

### DETAILED DESCRIPTION

The present disclosure is based in part upon the ability to bias the initial binding of a display library toward a target cell surface receptor binding site. Specifically, a receptor ligand or inhibitor is incorporated into the display library itself. Relying on the increased binding ability, via the interaction of both an individual antibodies complementary determining regions (CDRs) and the covalently attached ligand or inhibitor to stabilize the antibody: receptor complex to withstand an increasing stringency of the washing cycles used to remove any weak binders.

Thus, the present disclosure provided methods and compositions for generating binders, including antibodies, against a target protein. The target protein can be any protein of interest. For example, in any aspect or embodiment described herein, the target protein is a cell surface protein, an isolated protein, or a therapeutic protein target. Examples of therapeutic targets (e.g. targets that regulate the physiology of a cell and/or an organism) are known by those of skill in the art. The methods provided herein allow for the discovery of binders, including antibodies, that interact with a target peptide's active site, or regions that are adjacent to the active site. The methods further allow for the discovery of binders that interact with regions of the target protein that are distant from the active site of the target protein. In any aspect or embodiment described herein, the target protein is a G Protein-coupled Receptor (GPCR).

The methods provided by the disclosure therefore allow for the discovery of binders that can bind to any epitope of the target protein or peptide. In any aspect or embodiment described herein, the binders, once bound to the target protein or peptide, do not serve as an agonist or an antagonist. Alternatively, in any aspect or embodiment described herein, the binders once bound to the target protein or peptide, serve as an agonist or an antagonist. In any aspect or embodiment described herein, the binders can serve as allosteric or competitive inhibitors of a protein target. For example, in any aspect or embodiment described herein, the methods herein allow for the isolation of binders that can be used to modify the activity of a protein target. For example, in any aspect or embodiment described herein, the modification of the activity of a protein target includes the upregulation, downregulation, or ablation of the activity associated with a protein target.

The methods provided herein allow for the generation of binders (such as antibodies or antigen binding fragments thereof) that target a functional epitope of a target protein. Targeting of a functional epitope of a target protein allows the antibody to modulate the target protein's function. The ability to target a functional epitope allows the antibody to agonize or antagonize the target protein's function.

In any aspect or embodiment described herein, the methods provided herein use ligand-conjugated antigen binding region (e.g., antibody or antigen binding fragment thereof) libraries for the development of binders (e.g., antigen binding region or antibodies or an antigen binding fragment thereof) that can modulate a target protein's function.

In any aspect or embodiment described herein, the methods provided herein can also be utilized in combination with peptidomimetics or aptamers to find binders with high binding affinity. For example, in any aspect or embodiment described herein, a peptidomimetic is first discovered and isolated through means known in the art. In any aspect or embodiment described herein, the isolated peptidomimetic or aptamer can, for example, bind to a receptor or other peptide or protein. In any aspect or embodiment described herein, the isolated peptidomimetic or aptamer is a functional inhibitor or activator. In any aspect or embodiment described herein, the isolated peptidomimetic or aptamer once bound to its target does not inhibit or activate any function in the bound target.

In any aspect or embodiment described herein, the ligand (such as a peptidomimetic or aptamer) is enzymatically ligated to either the CDR antigen binding region (e.g., antibody or an antigen binding fragment thereof). In any aspect or embodiment described herein, the ligand (such as a peptidomimetic or aptamer) is enzymatically ligated to the framework region of the antigen binding region (e.g., antibody or an antigen binding fragment thereof).

Various methods of enzymatic ligation know in the art can be used. For example, in any aspect or embodiment described herein, ligation is accomplished through the use of sortases (recognizing “LPXTG”) or transglutaminases (recognizing a glutamine harbored by up to 6 specific amino acids on both sides).

Thus, the present disclosure describes a method, which has been termed Directed Ligand Assay (DLA) that can consistently target an immune response both in vitro and in vivo against a cell surface receptor regardless of the target's inherent immunogenicity in vivo.

Antibodies, or an antigen binding fragments thereof, developed using the methods of the present disclosure have application in a wide variety of fields, such as for example, in the development of vaccines, diagnostics, biosimilars, chimeric antigen receptor (CAR) T-cell therapy, therapeutics, bispecific antibodies, and multi-specific antibodies.

In any aspect or embodiment described herein, the methods described herein are utilized to generate binders (e.g., antibodies or an antigen binding fragment thereof) to linear or conformational epitopes.

Additionally, in any aspect or embodiment described herein, the methods described herein are utilized to identify any binding agent in a variety of display libraries. For example, in any aspect or embodiment described herein, the methods described herein are used in aptamer libraries to identify aptamers.

### Target Protein

Any protein can be a target protein. For example, the target protein is an integral membrane protein. Integral membrane proteins contain one or more regions which completely span the cell membrane. Often these molecules constitute important cell surface recognition or signaling molecules. Examples of integral membrane proteins include G protein-coupled receptors (GPCR), which classically have 7 transmembrane spanning regions, and ion channels and gates, whose pore-forming subunits typically have multiple transmembrane domains.

In any aspect or embodiment described herein, the target protein is an integral membrane protein (for example, a G-protein coupled receptor (GPCR)), an ion channel-coupled receptor, a viral receptor, or an enzyme-linked protein receptors. In any aspect or embodiment described herein, exemplary integral membrane proteins include receptor tyrosine kinases, insulin, select cell adhesion molecules (CAMs) (e.g., integrins, cadherins, neural cell adhesion molecules (NCAMs), selectins, glycophorin, rhodopsin, CD36, GPR30, glucose permease, gap junction proteins, and/or seipin).

In any aspect or embodiment described herein, the target protein is a GPCR. For example, in any aspect or embodiment described herein, the GPCR can be 5-Hydroxytryptamine receptors, Acetylcholine receptors (muscarinic), Adenosine receptors, Adhesion Class GPCRs, Adrenoceptors, Angiotensin receptors, Apelin receptor, Bile acid receptor, Bombesin receptors, Bradykinin receptors, Calcitonin receptors, Calcium-sensing receptor, Cannabinoid receptors, Chemerin receptor, Chemokine receptors, CXCR4, Cholecystokinin receptors, Class Frizzled GPCRs, Complement peptide receptors, Corticotropin-releasing factor receptors, Dopamine receptors, Endothelin receptors, G protein-coupled estrogen receptor, Formylpeptide receptors, Free fatty acid receptors, γ-aminobutyric acid type B (GABA_{B}) receptors, Galanin receptors, Ghrelin receptor, Glucagon receptor family, Glycoprotein hormone receptors, Gonadotrophin-releasing hormone receptors, G protein-coupled receptor 18 (GPR18), G protein-coupled receptor 55 (GPR55), G protein-coupled receptor 119 (GPR119), Histamine receptors, Hydroxycarboxylic acid receptors, Kisspeptin receptor, Leukotriene receptors, Lysophospholipid (LPA) receptors, Lysophospholipid (SiP) receptors, Melanin-concentrating hormone receptors, Melanocortin receptors, Melatonin receptors, Metabotropic glutamate receptors, Motilin receptor, Neuromedin U receptors, Neuropeptide FF receptors, neuropeptide AF receptors, Neuropeptide S receptor, Neuropeptide W receptors, neuropeptide B receptors, Neuropeptide Y receptors, Neurotensin receptors, Opioid receptors, Orexin receptors, Oxoglutarate receptor, P2Y receptors, Parathyroid hormone receptors, Platelet-activating factor receptor, Prokineticin receptors, Prolactin-releasing peptide receptor, Prostanoid receptors, Proteinase-activated receptors, pyroglutamylated RFamide peptide (QRFP) receptor, Relaxin family peptide receptors, Somatostatin receptors, Succinate receptor, Tachykinin receptors Thyrotropin-releasing hormone receptors, Trace amine receptor, Urotensin receptor, Vasopressin receptors, oxytocin receptors, vasoactive intestinal peptide (VIP) receptor, and/or pituitary adenylate-cyclase-activating polypeptide (PACAP) receptors.

In any aspect or embodiment described herein, the target protein is a lipase, a protease, a kinase, a sortase, or Cas9.

In any aspect or embodiment described herein, the target protein is a therapeutic protein target or a biological target. Therapeutic protein targets or biological targets that can be manipulated to achieve a certain physiological effect in an organism are known in the art. In any aspect or embodiment described herein, the therapeutic targets include or is, for example, anticoagulants, blood factors, bone morphogenetic proteins, engineered protein scaffolds, enzymes, growth factors, hormones, interferons, interleukins, or thrombolytics.

### Directed Ligand Antibodies

Recombinant antibodies (rAb), such as single chain variable fragments (scFv), have many attractive attributes compared to polyclonal antisera and monoclonal antibodies derived from hybridomas. Recombinant antibodies (i) are renewable through overexpression in the appropriate heterologous host, (ii) are easily stored and transferred as deoxyribonucleic acid (DNA), and (iii) can be genetically engineered as fusions to various enzymes, fluorescent proteins, and/or epitope tags. However, in vitro selection methods, such as phage display, yeast display, and ribosome display, have thus far been inefficient at meeting the need for customized antibodies that target membrane proteins. Several methods have been used to create antibody diversity in vitro, including cloning complementary DNA (cDNA) of the immune regions (a native approach) of either immunized or non-immunized vertebrate cells (to create a naive library), total synthesis of antibody CDR gene fragments with mixed-nucleotide synthesis, and a semi-synthetic approach, whereby a framework gene is synthesized and the diversity is generated by cloning a multitude of CDRs. Disadvantages to these library types include variable biophysical properties and expression levels using native libraries with heterogeneous frameworks and stop codons in mixed-nucleotide sequences in synthetic and semi-synthetic approaches. However, the total potential diversity with these libraries is still substantially higher (>10²³) than the diversity that can be sampled (typically 10¹¹-10¹² with phage libraries), and not all amino acids at a given CDR position will yield a folded antibody.

Described herein are methods to generate directed-ligand antibodies. Directed-ligand antigen binding region (e.g., antibody or antigen binding fragment thereof) makes use of ligand-target interactions to specifically target the functional epitope of a target protein. In any aspect or embodiment described herein, the functional epitope is an active site, a ligand binding site, or a catalytic site. In any aspect or embodiment described herein, the generation of a directed-ligand antibody includes: (1) providing a tethered antigen binding region or bifunctional compound (e.g., a tethered antibody or antigen binding fragment thereof) comprising an antigen binding region (e.g., antibody or antigen binding fragment thereof, such as ScFv) conjugated to a ligand that binds to a target protein; (2) generating a first library by randomizing one or more contact regions; (3) screening the first library to identify one or more tethered antigen binding regions or bifunctional compound with improved binding affinity to the functional epitope as compared to the ligand; (4) generating a second library by randomizing the ligand carrying region of the one or more tethered antigen binding regions or bifunctional compound (e.g., tethered antibodies or antigen binding fragment thereof) identified in the previous step; and (5) screening the second library to identify one or more antigen binding regions (e.g., antibodies or antigen binging fragment thereof) that bind to the functional epitope with the same or improved affinity in comparison to the ligand.

In any aspect or embodiment described herein, generating an antigen binding region (e.g., antibody or an antigen binding fragment thereof) against a target protein, comprising: (a) providing a bifunctional compound comprising an antigen binding region (e.g., antibody or antigen binding fragment thereof, such as ScFv) conjugated to a ligand that binds to the target protein; (b) generating a first antibody library by mutating one or more amino acids in the antigen binding regions; (c) screening the first library to identify one or more bifunctional compounds (e.g., one or more bifunctional compounds with an antigen binding region that binds to a target protein) with improved binding affinity to the target protein as compared to the ligand; (d) generating a second library by mutating the ligand (e.g., mutating one or more amino acids of a ligand that is a peptide or protein) of the one or more bifunctional compounds identified in step (c); (e) screening the second library to identify one or more antigen binding regions (e.g., antibodies or antigen binding fragments thereof) that bind to the target protein, thereby generating an antigen binding region (e.g., an antibody or an antigen binding fragment thereof) against the target protein (e.g., an epitope of a target protein).

### Ligand Receptor Pair Identification and Fusion of Tether Antibody to Ligand

The initial step in the generation of directed-ligand antibodies is the identification of a ligand-receptor pair. The ligand can be any compound. For example, in any aspect or embodiment described herein, the ligand can be a peptide or a small molecule compound. In any aspect or embodiment described herein, a small molecule is a compound having a molecular weight of below 2000, 1000, 900, 500, or 200 Daltons. In any aspect or embodiment described herein, the ligand is a polymer, DNA, ribonucleic acid (RNA), or a sugar. In any aspect or embodiment described herein, the ligand is a peptidomimetic or aptamer. In any aspect or embodiment described herein, methods for identifying a ligand-receptor pair include structural analysis of the antigen binding region in the peptide or protein of interest and/or in the ligand. Upon identification of a suitable ligand-receptor pair, in any aspect or embodiment described herein, the ligand is fused to an antigen binding region (e.g., antibody or antigen binding fragment thereof) template. In any aspect or embodiment described herein, the ligand can be fused or conjugated to a CDR, to the N-terminus of a light chain variable region, the C-terminus of a light chain variable region, the light chain constant region, the heavy chain variable region, the heavy chain constant region, he variable light chain framework region, or the variable heavy chain framework region.

There are numerous manners to fuse or conjugate the antigen binding region (e.g., antibody or antigen binding fragment thereof) template to the ligand. In any aspect or embodiment described herein, the antigen binding region (e.g., antibody or antigen binding fragment thereof) template can be any antibody or antibody fragment.

Any manner known in the art can be used to generate a fusion or conjugation between the antigen binding region (e.g., antibody or antigen binding fragment thereof) template and the ligand.

In any aspect or embodiment described herein, the framework region of the tether antibody is fused or conjugated to the ligand through a peptide bond, covalent bond, disulfide bond, or ester bond. In any aspect or embodiment described herein, sortases (recognizing “LPXTG”) or transglutaminases (recognizing a glutamine harbored by up to 6 specific amino acids on both sides) are used to fuse, conjugate, covalently link, or ligate the antigen binding region (e.g., antibody or antigen binding fragment thereof) to the ligand.

Sortases allow for site-specific fusion, conjugation, covalent linking, or ligation of the ligand to the antigen binding region (e.g., antibody or an antigen binding fragment thereof). Sortases are selective for specific for the C-terminal and N-terminal recognition motif amino acid sequence LPXTG, wherein X represents any amino acid, and the T and the G in the substrate can be connected using a peptide bond or an ester linkage. In any aspect or embodiment described herein, a sortase recognition sequence is engineered to allow for fusion of the antigen binding region (e.g., antibody or an antigen binding fragment thereof) to the ligand.

### First Library Generation

In any aspect or embodiment described herein, a first antibody library is generated. Generation of the first antibody library can be made through any method known in the art. In any aspect or embodiment described herein, a first antibody library is generated by mutating one or more amino acids in the antigen binding regions of the antigen binding region. In any aspect or embodiment described herein, a first library is generated by randomizing one or more contact regions of the antigen binding region adjacent to a binding site between the ligand and the functional epitope. In this manner, the one or more contact regions are randomized without altering the ligand carrying region of the tether antibody or bifunctional compound template. In any aspect or embodiment described herein, the contact regions can be one or more complementarity determining regions (CDRs) that are selected for mutagenesis. In any aspect or embodiment described herein, stop codons and/or restriction enzyme cleavage sites are incorporated into the selected CDRs. In any aspect or embodiment described herein, the stop codons and restriction enzyme cleavage sites are replaced through site-directed mutagenesis. Any kind of site-directed mutagenesis known in the art can be used. In any aspect or embodiment described herein, Kunkel-based mutagenesis is used to replace incorporated stop codons and/or restriction enzyme recognition sites with tri-oligonucleotides that encode naturally distributed sets of residues at selected CDR positions. In any aspect or embodiment described herein, the resulting DNA template is then amplified.

Any kind of library amplification methods known in the art can be used for library amplification. In any aspect or embodiment described herein, a sequence of interest may be amplified using a pair of oligonucleotides, of which one oligonucleotide is a protected oligonucleotide and the other is a non-protected oligonucleotide. In any aspect or embodiment described herein, the sequence of interest may be amplified using such an oligonucleotide pair by an amplification reaction (such as polymerase chain reaction (PCR), error-prone PCR, isothermal amplification, or rolling circle amplification). In any aspect or embodiment described herein, rolling circle amplification (RCA) is used. In any aspect or embodiment described herein, error-prone rolling circle amplification is used. In any aspect or embodiment described herein, the RCA amplifies the library by about between about 50-fold and about 150-fold (e.g., about 50, about 55, about 60, about 65, about 70, about 75, about 80, about 85, about 90, about 95, about 100, about 105, about 110, about 115, about 120, about 125, about 130, about 135, about 140, about 145, about 150, any values in between, and a range from any combination of the values). In any aspect or embodiment described herein, the RCA amplifies the library by about 100-fold. In any aspect or embodiment described herein, the RCA amplified library is linearized and re-circularized.

In any aspect or embodiment described herein, the ligand-antibody library is introduced into any suitable cell known in the art. In any aspect or embodiment described herein, the cell is an archaeal cell, prokaryotic cell, bacterial cell, fungal cell, or eukaryotic cell. In any aspect or embodiment described herein, the cell is a yeast cell, plant cell, or animal cell. In any aspect or embodiment described herein, the cell is an Escherichia coli cell or Saccharomyces cerevisiae cell. In any aspect or embodiment described herein, the cell strain is an electro-competent cell or chemical competent cell. In any aspect or embodiment described herein, the library is transformed into DH5a, JM109, C600, HB101, or TG1. In any aspect or embodiment described herein, the library is transformed into TG1 cells. In any aspect or embodiment described herein, the plurality of tether antibodies or bifunctional compounds are expressed as a soluble protein in the periplasm.

In any aspect or embodiment described herein, the first library has a diversity of between about 10⁷ and about 10¹⁴ (e.g., about 10⁷, about 10⁸, about 10⁹, about 10¹⁰, about 10¹¹, about 10¹², about 10¹³, about 10¹⁴, or a range from any combination of the values) unique ligand-antibodies. In any aspect or embodiment described herein, the first library has a diversity of between at least 10⁸ and about 10¹² (e.g., about 10⁸, about 10⁹, about 10¹⁰, about 10¹¹, 10¹², or a range from any combination of the values).

In any aspect or embodiment described herein, the antibody library is a phage library. The phage used in the phage library can be any phage. In any aspect or embodiment described herein, the phage used is M13, fd filamentous phage, T4, T7, or .lamda phage. In any aspect or embodiment described herein, the phage used in the phage library is M13 phage. In any aspect or embodiment described herein, the tether antibody or bifunctional compound templates are expressed on a selected phage coat protein. Suitable phage coat proteins are known in the art. In any aspect or embodiment described herein, the phage coat protein is gpIII.

### Screening and Validating the First Library

In any aspect or embodiment described herein, the first library is screened to identify one or more bifunctional compounds (e.g., one or more bifunctional compounds with an antigen binding region that binds to a target protein) with improved binding affinity to the target protein as compared to the ligand. Any method known in the art can be used to screen the first library for binding to the ligand. In any aspect or embodiment described herein, an emulsion whole cell-based library screening method is used. Whole cell screening methods (e.g. whole cell panning) are described in, e.g., United States patent application publication number 2015/03322150, the contents of which are incorporated by reference herein in its entirety. In any aspect or embodiment described herein, the whole cell screening method includes, for example, the creation of an emulsion of expression cells (e.g., E. coli) transduced with the bifunctional compound (or ligand-antigen binding region) phage library incubated with cells or beads that display the target protein or antigen of interest. In any aspect or embodiment described herein, during an overnight incubation process, bifunctional compound (or ligand-antigen binding region) displaying phages are secreted from the expression cell (e.g., E. coli) and attach to the antigen presenting cells or beads. In any aspect or embodiment described herein, subsequent processing includes the addition of labeled antibodies that attach to the phage, and subsequent FACS sorting to isolate the bifunctional compound (or ligand-antigen binding region) displaying phage that have bound to the antigens displayed on the whole cell or beads. In any aspect or embodiment described herein, the library is processed for multiple rounds of whole cell screening. In any aspect or embodiment described herein, whole cell screening is performed between about 3 to 8 times (e.g., 3, 4, 5, 6, 7, or 8). In any aspect or embodiment described herein, whole cell screening is performed about 3 times. In any aspect or embodiment described herein, multiple rounds of whole cell screening results in the isolation of more specific epitope binding antigen binding regions (e.g., antibodies or antigen binding fragments thereof).

In any aspect or embodiment described herein, the isolated bifunctional compounds (or ligand-antigen binding regions) are further validated by use of enzyme-linked immunosorbent assay (ELISA), a functional competition assay, or a combination thereof. In any aspect or embodiment described herein, the competition assay includes, for example, a competition assay using free ligands. These further validations are intended to ascertain that the binding of the antigen binding region (e.g., antibody or antigen binding fragment thereof) to the target protein is improved in comparison to the binding of ligand alone. In any aspect or embodiment described herein, one or more methods to enhance enrichment for whole cell panning are used. For example, in any aspect or embodiment described herein, enrichment for whole cell panning is accomplished via induced hexamerization. In any aspect or embodiment described herein, hexamerization is performed by genetically linking hexamerizing protein (TH7) to cytoplasmic or extracellular domain of a membrane protein to enhance avidity. In any aspect or embodiment described herein, the creation of an OmpA-TH7-linker-FLAG on the cells' outer membrane enriches whole cell panning.

### Second Library Generation and Screening

In any aspect or embodiment described herein, following the isolation of screened and validated binders from the first library, a second library is generated. In any aspect or embodiment described herein, the second library is a phage library. The purpose of the second library is to eliminate, reduce, and/or phase out the affinity contributed by the ligand in the isolated bifunctional compounds (or ligand-antigen binding region). In any aspect or embodiment described herein, to this end, mutations will be introduced into the ligand, which was not mutated in the first library.

In any aspect or embodiment described herein, two randomization strategies are used, and the end products are combined to produce the second library.

In any aspect or embodiment described herein, the first randomization strategy introduces about 1 to about 10% mutation rate (e.g., about 1.0, about 1.5, about 2.0, about 2.5. about 3.0, about 3.5, about 4.0, about 4.5, about 5.0, about 5.5, about 6.0, about 6.5, about 7.0, about 7.5, about 8.0, about 8.5, about 9.0, about 9.5, about 10, any values in between, or a range from any combination of the values) to the ligand and/or its flanking region using any method known in the art. In any aspect or embodiment described herein, the first randomization strategy introduces about 2 to about 5% (e.g., about 2.0, about 2.5, about 3.0, about 3.5, about 4.0, about 4.5, about 5.0, any values in between, or a range from any combination of the values) mutation rate to the ligand and/or its flanking region using any method known in the art. In any aspect or embodiment described herein, the mutations are introduced by error-prone PCR.

In any aspect or embodiment described herein, the second randomization strategy introduces segmental randomization. In any aspect or embodiment described herein, segmental randomization uses an NNK randomization scanning window of 9 nucleotides (or 3 amino acids) that is applied on the ligand and at −4aa and +4aa of the flanking regions.

Any kind of library amplification methods known in the art can be used for amplification of the second library. In any aspect or embodiment described herein, a sequence of interest may be amplified using a pair of oligonucleotides, of which one oligonucleotide is a protected oligonucleotide and the other is a non-protected oligonucleotide. In any aspect or embodiment described herein, the sequence of interest is amplified using such an oligonucleotide pair by an amplification reaction (such as PCR, error-prone PCR, isothermal amplification, or rolling circle amplification). In any aspect or embodiment described herein, rolling circle amplification (RCA) is used. In any aspect or embodiment described herein, error-prone rolling circle amplification is used. In any aspect or embodiment described herein, the RCA amplifies the library by about between about 50-fold and about 150-fold (e.g., about 50, about 55, about 60, about 65, about 70, about 75, about 80, about 85, about 90, about 95, about 100, about 105, about 110, about 115, about 120, about 125, about 130, about 135, about 140, about 145, about 150, any values in between, or a range from any combinations of the values). In any aspect or embodiment described herein, the RCA amplifies the library by about 100-fold. In any aspect or embodiment described herein, the RCA amplified library is linearized and re-circularized.

The second library can be introduced into any suitable cell known in the art. The cell may be an archaeal cell, prokaryotic cell, bacterial cell, fungal cell, or eukaryotic cell. The cell may be a yeast cell, plant cell, or animal cell. In some instances, the cell may be an E. coli cell or S. cerevisiae cell. The cell strain can be any electro- or chemical competent cell. In some embodiments, the library can be transformed into DH5a, JM109, C600, HB101, or TG1. In some embodiments, the library is transformed into TG1 cells.

In some embodiments, the second library has a diversity of between about 10⁷ and about 10¹⁴ (e.g., about 10⁷, about 10⁸, about 10⁹, about 10¹⁰, about 10¹¹, about 10¹², about 10¹³, about 10¹⁴, or a range from any combination of the values) unique ligand-antibodies. In some embodiments, the second library has a diversity of between at least 10⁸ and about 10¹² (e.g., about 10⁸, about 10⁹, about 10¹⁰, about 10¹¹, about 10¹², or a range from any combination of the values).

In any aspect or embodiment described herein, the second library is screened by the whole cell-based library screening method. In any aspect or embodiment described herein, the library is processed for multiple rounds of whole cell screening. In any aspect or embodiment described herein, whole cell screening is performed between about 3 to 8 times (e.g., 3, 4, 5, 6, 7, or 8). In any aspect or embodiment described herein, whole cell screening is performed 3 times. In any aspect or embodiment described herein, multiple rounds of whole cell screening results in the isolation of more specific epitope binding antigen binding regions (e.g., antibodies or antigen binding fragments thereof).

In any aspect or embodiment described herein, the second library binders are further validated by use of ELISA, functional competition assay, or a combination thereof. In any aspect or embodiment described herein, the isolated clones that have an ELISA signal greater than 2-fold over background will be expressed in E. coli and purified by metal chromatography. In any aspect or embodiment described herein, a further functional validation is performed on the isolated second library binders. Any method known in the art can used to validate the second library binders.

### Linker

In any aspect or embodiment described herein, the methods herein use a ligand tethered to an antigen binding region (e.g., antibody or antigen binding fragment thereof) by a linker. In any aspect or embodiment described herein, the linker is a peptide, polypeptide, or protein.

In any aspect or embodiment described herein, the length of the peptide, polypeptide, or protein linker is between about 3 to about 50 (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids. In any aspect or embodiment described herein, the length of the linker is between about 3 to about 21 (e.g., 3, 4, 5, 6, 7, 8, 9, 10, 11, 12, 13, 14, 15, 16, 17, 18, 19, 20, or 21) amino acids.

In any aspect or embodiment described herein, the linker is optimized to enhance binding of the antigen binding region (e.g., antibody or antigen binding fragment thereof) and/or ligand. In any aspect or embodiment described herein, the length of the linker is optimized by screening a mini-library that includes a plurality of linker peptides having various lengths. In any aspect or embodiment described herein, the mini-library is constructed from single-stranded DNA (ssDNA) from the template that had the best affinity in cell ELISA validation. In any aspect or embodiment described herein, an oligo set that carries a random length central region of about between about 3 and about 80 nucleotides (e.g., 3, 4, 5, 6, 7, 8, 9, 10, 15, 20, 25, 30, 35, 40, 45, 50, 55, 60, 65, 66, 67, 68, 69, 70, 75, 80, any number in between, or a range from any combinations of the numbers) is used. In any aspect or embodiment described herein, an oligo set that carries a random length central region of between about 6 and about 66 nucleotides (e.g., 6, 7, 8, 9, 10, 15, 20, 25, 30, 35, 40, 45, 50, 55, 60, 65, 66, any number in between, or a range from any combinations of the numbers) is used. In any aspect or embodiment described herein, the oligo is flanked by two cognate regions that will be annealed to the template ssDNA, each cognate region independently about 15 to about 30 (e.g., 15, 16, 17, 18, 19, 20, 21, 22, 23, 24, 25, 26, 27, 28, 29, or 30) nucleotides. In any aspect or embodiment described herein, the oligo set is flanked by two cognate regions that will be annealed to the template ssDNA, each cognate region being 20 nucleotides. In any aspect or embodiment described herein, the resulting mini library of phage that display the scFv-ligand fusion with varied connecting loop lengths is made by Kunkel mutagenesis. Other methods known in the art can be used to vary the linker length. In any aspect or embodiment described herein, the mini library with varied linker lengths is enriched for the strongest binders by whole cell panning, and the isolated binders are sequenced.

In any aspect or embodiment described herein, the linker has one or more cleavable region. For example, in any aspect or embodiment described herein, the connecting loop has an enzyme cleavage site (for example, a thrombin cleavage site (“GRG”)).

### Antigen Binding Region

In any aspect or embodiment described herein, the antigen binding region (or tethered antigen binding region) is a whole antibody or immunoglobulin or an antigen binding fragment of an antibody. In any aspect or embodiment described herein, the antigen binding region (or tethered antigen binding region) is an scFv, Fab, Fab′, or IgG.

In any aspect or embodiment described herein, the antigen binding region (or tethered antigen binding region) is a single chain variable fragment (scFv) comprising a heavy chain variable region, a light chain variable region, and a scFv peptide linker (e.g., 10 to about 25 amino acids, or 10, 11, 12, 13, 14, 15, 16, 17, 18, 19, 20, 21, 22, 23, 24, or 25 amino acids) that links the heavy chain variable region and the light chain variable region. In any aspect or embodiment described herein, the antigen binding region (or tethered antigen binding region) is a single chain variable fragment (scFv) comprising a heavy chain variable region, a light chain variable region, and a scFv peptide linker (e.g., 10 to about 25 amino acids, or 10, 11, 12, 13, 14, 15, 16, 17, 18, 19, 20, 21, 22, 23, 24, or 25 amino acids) that links the heavy chain variable region and the light chain variable region, wherein the ligand is conjugated to the scFv peptide linker.

In any aspect or embodiment described herein, the antigen binding region (or tethered antigen binding region) is a single chain variable fragment (scFv) comprising a heavy chain variable region fused to a light chain variable region via a scFv peptide linker (e.g., 10 to about 25 amino acids, or 10, 11, 12, 13, 14, 15, 16, 17, 18, 19, 20, 21, 22, 23, 24, or 25 amino acids). In any aspect or embodiment described herein, the ligand is conjugated to the scFv peptide linker. In any aspect or embodiment described herein, the antigen binding region (or tethered antigen binding region) is a single chain variable fragment (scFv) comprising a heavy chain variable region fused to a light chain variable region via a scFv peptide linker (e.g., 10 to about 25 amino acids, or 10, 11, 12, 13, 14, 15, 16, 17, 18, 19, 20, 21, 22, 23, 24, or 25 amino acids), wherein the ligand is conjugated to the scFv peptide linker.

In any aspect or embodiment described herein, the terminal product of the method described herein is an antibody free of natural ligand, enabling the creation of an agonist or an antagonist. In any aspect or embodiment described herein, the ligands are not limited to peptides or proteins. For example, in any aspect or embodiment described herein, through the use of artificial disulfide bond crosslinking, any molecule that carries a free thiol can be linked and applied to the initial screen. Since the artificial bond is phased out in the second-round screening, the ligand carrying regions are also not limited to the CDRs. Thus, in any aspect or embodiment described herein, the ligand carrying region may be outside of the CDR regions, and may be located, for example, in the framework region.

In any aspect or embodiment described herein, the ligand is not included in the final antigen binding region (e.g., antibody or antigen binding fragment thereof) product. Thus, the affinity of the final product does not rely on the affinity of the ligand. Furthermore, in any aspect or embodiment described herein, weaker binding ligands may perform equivalently for initial tethering and directing purposes.

In any aspect or embodiment described herein, an antibody, or antigen binding fragment thereof, of the present disclosure may be multi-specific (e.g., bispecific). In any aspect or embodiment described herein, the antibody, or antigen binding fragment thereof, of the disclosure is mammalian (e.g., human or mouse), humanized, chimeric, recombinant, synthetically produced, or naturally isolated. In any aspect or embodiment described herein, the antigen binding region of the present disclosure includes, is, or is part of, without limitation, IgG (e.g., IgG1, IgG2, IgG3, and IgG4), IgM, IgA (e.g., IgAQ1, IgA2, and IgAsec), IgD, IgE, Fab, Fab′, Fab′2, F(ab′)2, Fd, Fv, Feb, scFv, scFv-Fc, or surface molecularly imprinted polymer (SMIP) binding moiety. In any aspect or embodiment described herein, the antigen binding region is, is part of, or includes an scFv. In any aspect or embodiment described herein, the scFv includes, for example, a flexible linker allowing the scFv to orient in different directions to enable antigen binding. In any aspect or embodiment described herein, the antigen binding region is, is part of, or includes a cytosol-stable scFv or intrabody that retains its structure and function in the reducing environment inside a cell. In any aspect or embodiment described herein, the scFv is converted to an IgG or a chimeric antigen receptor according to the methods known in the art and described herein.

In most mammals, including humans, whole antibodies have at least two heavy (H) chains and two light (L) chains connected by disulfide bonds. Each heavy chain consists of a heavy chain variable region (VH) and a heavy chain constant region (CH). The heavy chain constant region consists of three domains (C_{H1}, C_{H2}, and C_{H3}) and a hinge region between C_{H1} and C_{H2}. Each light chain consists of a light chain variable region (VL) and a light chain constant region (CL). The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen.

Antibodies of the present disclosure include all known forms of antibodies and other protein scaffolds with antibody-like properties. For example, in any aspect or embodiment described herein, the antibody is a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. In any aspect or embodiment described herein, the antibody has any of the following isotypes: IgG (e.g., IgG1, IgG2, IgG3, and IgG4), IgM, IgA (e.g., IgAQ1, IgA2, and IgAsec), IgD, or IgE.

In any aspect or embodiment described herein, an antibody fragment may include one or more segments derived from an antibody. A segment derived from an antibody may retain the ability to specifically bind to a particular antigen. In any aspect or embodiment described herein, an antibody fragment is a Fab, Fab′, Fab′2, F(ab′)2, Fd, Fv, Feb, scFv, or SMIP. In any aspect or embodiment described herein, an antibody fragment is a diabody, triabody, affibody, nanobody, aptamer, domain antibody, linear antibody, single-chain antibody, or any of a variety of multi-specific antibodies that may be formed from antibody fragments.

In any aspect or embodiment described herein, examples of antibody fragments include: (i) a Fab fragment: a monovalent fragment consisting of VL, VH, CL, and C_{H1} domains; (ii) a F(ab′)2 fragment: a bivalent fragment comprising two Fab fragments linked by a disulfide bridge at the hinge region; (iii) a Fd fragment: a fragment consisting of VH and C_{H1} domains; (iv) an Fv fragment: a fragment consisting of the VL and VH domains of a single arm of an antibody; (v) a dAb fragment: a fragment including VH and VL domains; (vi) a dAb fragment: a fragment that is a VH domain; (vii) a dAb fragment: a fragment that is a VL domain; (viii) an isolated complementarity determining region (CDR); and (ix) a combination of two or more isolated CDRs which is optionally joined by one or more synthetic linkers. Furthermore, although the two domains of the Fv fragment, VL and VH, are coded for by separate genes, they can be joined, using recombinant methods, e.g., by a synthetic linker that enables them to be expressed as a single protein, of which the VL and VH regions pair to form a monovalent binding moiety (known as a single chain Fv (scFv)). Antibody fragments may be obtained using conventional techniques known to those of skill in the art, and may, in some instances, be used in the same manner as intact antibodies. In any aspect or embodiment described herein, an antigen-binding fragment is produced by recombinant DNA techniques or by enzymatic or chemical cleavage of intact immunoglobulins. In any aspect or embodiment described herein, an antibody fragment further includes any of the antibody fragments described above with the addition of additional C-terminal amino acids, N-terminal amino acids, amino acids separating individual fragments, or a combination thereof.

In any aspect or embodiment described herein, the antibody is a chimeric antibody. An antibody may be referred to as chimeric if it includes one or more antigen-determining regions or constant regions derived from a first species and one or more antigen-determining regions or constant regions derived from a second species. Chimeric antibodies may be constructed, e.g., by genetic engineering. A chimeric antibody may include immunoglobulin gene segments belonging to different species (e.g., from a mouse and a human).

In any aspect or embodiment described herein, an antibody may be a human antibody. A human antibody refers to a binding moiety having variable regions in which both the framework and CDR regions are derived from human immunoglobulin sequences. Furthermore, if the antibody contains a constant region, the constant region also is derived from a human immunoglobulin sequence. In any aspect or embodiment described herein, a human antibody includes amino acid residues not identified in a human immunoglobulin sequence (e.g., one or more sequence variations, such as mutations). In any aspect or embodiment described herein, one or more variations or additional amino acids are introduced (e.g., by human manipulation). In any aspect or embodiment described herein, a human antibody of the present disclosure is not chimeric.

An antibody may be humanized, meaning that an antibody that includes one or more antigen-determining regions (e.g., at least one CDR) substantially derived from a non-human immunoglobulin or antibody is manipulated to include at least one immunoglobulin domain substantially derived from a human immunoglobulin or antibody. In any aspect or embodiment described herein, the antibody is a humanized antibody. An antibody may be humanized using the conversion methods described herein, for example, by inserting antigen-recognition sequences from a non-human antibody encoded by a first vector into a human framework encoded by a second vector. For example, the first vector may include a polynucleotide encoding the non-human antibody (or a fragment thereof) and a site-specific recombination motif, while the second vector may include a polynucleotide encoding a human framework and a site-specific recombination complementary to a site-specific recombination motif on the first vector. The site-specific recombination motifs may be positioned on each vector such that a recombination event results in the insertion of one or more antigen-determining regions from the non-human antibody into the human framework, thereby forming a polynucleotide encoding a humanized antibody.

In any aspect or embodiment described herein, a ligand free antibody is converted from scFv to an IgG (e.g., IgG1, IgG2, IgG3, and IgG4). There are various methods in the art for converting scFv fragments to IgG. One such method of converting scFv fragments to IgG is disclosed in United States patent application publication number 2016/0362476, the contents of which are incorporated by reference herein in its entirety.

### Binding Affinity

Binding affinity for antibodies and antibody fragments are determined through various methods known in the art. For example, binding affinity can be determined by the Scatchard analysis of Munson and Pollard, Anal. Biochem., 107:220 (1980). Another method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity and is equal to the dissociation constant K_{d}. (See, generally, Davies et al. (1990) Annual Rev Biochem 59:439-473).

In any aspect or embodiment described herein, the binding affinity (K_{d}) of the antigen binding region (e.g., antibody or antigen binding fragment thereof) to the target protein is less than about 100 μM, about 10 μM, about 1 μM, about 100 nM, about 10 nM, about 1 nM, about 100 μM, about 10 pM, or about 1 pM. In any aspect or embodiment described herein, the binding affinity (K_{d}) of the antigen binding region (e.g., antibody or antigen binding fragment thereof) to the target protein is about 1 pM to about 50 nM (e.g., about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 1 pM, about 2 pM, about 3 pM, about 4 pM, about 5 pM, about 6 pM, about 7 pM, about 8 pM, about 9 pM, about 10 pM, about 15 pM, about 20 pM, about 25 pM, about 30 pM, about 35 pM, about 40 pM, about 45 pM, about 50 pM, any values in between, or a range from any combination of the values). In any aspect or embodiment described herein, the binding affinity (K_{d}) of the antigen binding region (e.g., antibody or antigen binding fragment thereof) to the target protein is about 1 pM to about 15 nM (e.g., about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, any values in between, or a range from any combination of the values).

### Methods of Using Directed Ligand Antibodies

It would be readily apparent to one skilled in the art that the directed ligand binder or antigen binding region (e.g., antibody or antigen binding fragment thereof) produced by the methods described herein are useful in a variety of diagnostic and therapeutic applications. For example, in the development of vaccines, diagnostics, biosimilars, CAR-T cell therapy, therapeutics, bispecific antibodies, and multi-specific antibodies.

A further aspect of the present disclosure provides a method of treating, preventing, and/or ameliorating at least one symptom of a disease or disorder (e.g., pneumonia, a viral infection, a bacterial infection, a coronavirus infection (such as SARS-CoV-2), a coronavirus-related disease (e.g., Coronavirus disease 2019 (COVID-19)), a human immunodeficiency virus (HIV) infection, an ebolavirus infection (e.g., an Ebola virus (EBOV) infection, Sudan virus (SUDV) infection, Bundibugyo virus (BDBV) infection, Reston virus (RESTV) infection, Tai Forest virus (TAFV) infection, etc.) in a subject in need thereof. The method comprises: providing a subject in need thereof; and administering an effective amount of the pharmaceutical composition or formulation described herein, wherein the antibody effectuates the prevention, treatment, or amelioration of at least one symptom of the disease or disorder.

An additional aspect of the present disclosure provides pharmaceutical compositions or formulations comprising the directed ligand binder (e.g., antigen binding region or antibody or antigen binding fragment thereof). Pharmaceutical compositions or formulations described herein further comprises an effective amount of an excipient (e.g., an effective amount of a pharmaceutically acceptable excipient) or carrier (e.g., an effective amount of a pharmaceutically acceptable carrier). As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the binder (e.g., antigen binding region, or antibody or antigen binding fragment thereof) of the present disclosure, use thereof in the compositions is contemplated. Supplementary active compounds can also be incorporated into the compositions.

The description provides methods for preparing pharmaceutical compositions or formulations. Such methods comprise formulating an effective amount of a pharmaceutically acceptable carrier or excipient with one or more antibody as described herein. Such compositions or formulations can further include additional active agents as described above. Thus, the present disclosure further describes methods for preparing a pharmaceutical composition or formulation.

A pharmaceutical composition or formulation of the present disclosure is formulated to be compatible with its intended route of administration. Examples of routes of administration include parenteral (e.g., intravenous, intradermal, subcutaneous, intramuscular, intraperitoneal, intranodal, and intrasplenic) administration. Solutions or suspensions used for parenteral, intradermal, or subcutaneous application can include the following components: a sterile diluent (such as water for injection, saline solution, fixed oils, polyethylene glycols, glycerine, propylene glycol or other synthetic solvents); antibacterial agents (such as benzyl alcohol); antioxidants (such as ascorbic acid or sodium bisulfate); chelating agents (such as ethylenediamine-tetraacetic acid); buffers (such as acetates, citrates or phosphates and agents for the adjustment of tonicity such as sodium chloride or dextrubinrubi). pH can be adjusted with acids or bases, such as hydrochloric acid or sodium hydroxide. The parenteral preparation can be enclosed in ampules, disposable syringes or multiple dose vials made of glass or plastic.

Pharmaceutical compositions or formulations suitable for injectable use, in any aspect or embodiment described herein, include sterile aqueous solutions (where water soluble) or dispersions and sterile powders for the extemporaneous preparation of sterile injectable solutions or dispersions. In any aspect or embodiment described herein, for intravenous administration, suitable carriers include physiological saline, bacteriostatic water, Cremophor EL (BASF; Parsippany, N.J.) or phosphate buffered saline (PBS). In all cases, the composition or formulation must be sterile and should be fluid to the extent that easy syringability exists. It must be stable under the conditions of manufacture and storage and must be preserved against the contaminating action of microorganisms such as bacteria and fungi. In any aspect or embodiment described herein, the carrier can be a solvent or dispersion medium containing, for example, water, ethanol, polyol (for example, glycerol, propylene glycol, and liquid polyethylene glycol, and the like), and suitable mixtures thereof. In any aspect or embodiment described herein, the proper fluidity can be maintained, for example, by the use of a coating (such as lecithin), by the maintenance of the required particle size in the case of dispersion, by the use of surfactants, or a combination thereof. In any aspect or embodiment described herein, prevention of the action of microorganisms is achieved by various antibacterial and antifungal agents (for example, chlorobutanol, phenol, ascorbic acid, and the like). In any aspect or embodiment described herein, it will be preferable to include isotonic agents (for example, sugars, polyalcohols (such as mannitol or sorbitol), or sodium chloride) in the composition or formulation. In any aspect or embodiment described herein, prolonged absorption of the injectable compositions or formulations can be brought about by including in the composition or formulation an agent which delays absorption (for example, aluminum monostearate, gelatin, or a combination thereof).

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is especially advantageous to formulate parenteral compositions or formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure are dictated by and directly dependent on the unique characteristics of the binder (e.g., antigen binding region, or antibody or antigen binding fragment thereof) and the particular therapeutic effect to be achieved, and the limitations inherent in the art of compounding, such a binder or an antibody or antigen binding fragment thereof, for the treatment of subjects.

In any aspect or embodiment of the methods provided herein, the method can further include the step of administering a dosage from about 100 ng to about 200 mg of a therapeutic or pharmaceutical composition or formulation as described herein. In any aspect or embodiment described herein, e.g., in human, the pharmaceutical composition or formulation as described herein may contain mannitol as carrier, and the composition or formulation is administered from 10 μg to 200 mg, preferably 20 to 100 mg, in a single administration.

In any aspect or embodiment described herein, preferred pharmaceutically acceptable carriers comprise, for example, xanthan gum, locust bean gum, galactose, other saccharides, oligosaccharides and/or polysaccharides, starch, starch fragments, dextrins, British gum, or mixtures thereof. Advantageously, in any aspect or embodiment described herein, the pharmaceutically acceptable carrier is of natural origin. In any aspect or embodiment described herein, the pharmaceutically acceptable carrier is, or further comprises, an inert saccharide diluent selected from a monosaccharide or disaccharide (e.g., mannitol).

In any aspect or embodiment described herein, the composition further comprises at least one stabilizer (e.g., one or more of salt(s), saccharide(s), and/or amino acid(s)). In any aspect or embodiment described herein, the composition further comprises at least one surfactant. In any aspect or embodiment described herein, the composition further comprises at least one buffering agent.

The term “surfactant” as used herein refers to a pharmaceutically acceptable, surface-active agent. For example, in any aspect or embodiment described herein, a non-ionic surfactant is used. Examples of pharmaceutically acceptable surfactants include, but are not limited to, polyoxyethylen-sorbitan fatty acid esters (Tween®), polyoxyethylene alkyl ethers (Brij), alkylphenylpolyoxyethylene ethers (Triton X), polyoxyethylenepolyoxypropylene copolymers (Poloxamer, Pluronic), sodium dodecyl sulphate (SDS), or mixtures thereof. In any aspect or embodiment described herein, preferred polyoxyethylene-sorbitan fatty acid esters are polysorbate 20 (polyoxyethylene sorbitan monolaureate, sold under the trademark Tween 20™) or polysorbate 80 (polyoxyethylene sorbitan monooleate, sold under the trademark Tween 80™). In any aspect or embodiment described herein, preferred polyethylene-polypropylene copolymers are those sold under the names Pluronic® F68 or Poloxamer 188™. In any aspect or embodiment described herein, preferred polyoxyethylene alkyl ethers are those sold under the trademark Brij™. In any aspect or embodiment described herein, preferred alkylphenylpolyoxyethylene ethers are sold under the tradename Triton X, most preferred is p-tert-octylphenoxy polyethoxyethanol (sold under the tradename Triton X-100™). In any aspect or embodiment described herein, preferred surfactants for use in the present invention are polyoxyethylen-sorbitan fatty acid esters, preferably polysorbate 20 or polysorbate 80, most preferably polysorbate 20. In any aspect or embodiment described herein, another preferred surfactant is Poloxamer 188™.

The term “buffering agent” as used herein refers to a pharmaceutically acceptable excipient, which stabilizes the pH of a pharmaceutical preparation. Suitable buffers are well known in the art and can be found in the literature. In any aspect or embodiment described herein, pharmaceutically acceptable buffers comprise, but are not limited to histidine-buffers, citrate-buffers, succinate-buffers, acetate-buffers, arginine-buffers, phosphate-buffers, or mixtures thereof. Buffering agents are thus histidine salts, citrate salts, succinate salts, acetate salts, malate salts, phosphate salts and lactate salts. Buffering agents of particular interest comprise L-histidine or mixtures of L-histidine and L-histidine hydrochloride or L-histidine acetate with pH adjustment with an acid or a base known in the art. In any aspect or embodiment described herein, the above-mentioned buffers are used in an amount of about 5 mM to about 100 mM, particularly of about 10 mM to about 30 mM, and more particularly of about 20 mM. Independently from the buffer used, in any aspect or embodiment describe herein, the pH can be adjusted to a value in the range from about 4.5 to about 7.0, and particularly to a value in the range from about 5.0 to about 6.0, and most particularly to pH 6.0.+−0.0.03 with an acid or a base known in the art, e.g. hydrochloric acid, acetic acid, phosphoric acid, sulfuric acid and citric acid, sodium hydroxide and potassium hydroxide.

The term “stabilizer” as used herein refers to a pharmaceutical acceptable excipient, which protects the active pharmaceutical ingredient and/or the formulation from chemical and/or physical degradation during manufacturing, storage and application. In any aspect or embodiment described herein, stabilizers include, but are not limited to, saccharides, amino acids, polyols (e.g. mannitol, sorbitol, xylitol, dextran, glycerol, arabitol, propylene glycol, polyethylene glycol), cyclodextrines (e.g. hydroxypropyl-.beta.-cyclodextrine, sulfobutyl-ethyl-.beta.-cyclodextrine, .beta.-cyclodextrine), polyethylenglycols (e.g. PEG 3000, PEG 3350, PEG 4000, PEG 6000), albumines (human serum albumin (HSA), bovine serum albumin (BSA)), salts (e.g. sodium chloride (saline), magnesium chloride, calcium chloride), and/or chelators (e.g. EDTA). In any aspect or embodiment described herein, the stabilizer is selected from the group consisting of saccharides, polyols, and amino acids. In any aspect or embodiment described herein, the one or more stabilizers is present in the formulation in an amount of about 10 mM to about 500 mM, particularly in an amount of about 140 to about 250 mM, and more particularly in an amount of about 210 mM to about 240 mM. For example, in any aspect or embodiment described herein, sucrose or trehalose are used as stabilizers in an amount of about 220 mM to about 240 mM.

The term “saccharide” as used herein includes monosaccharides and oligosaccharides. A monosaccharide is a monomeric carbohydrate which is not hydrolysable by acids, including simple sugars and their derivatives, e.g. aminosugars. Saccharides are usually in their D conformation. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a linear chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. Preferred saccharides for use in the present invention are sucrose and trehalose (i.e. α,α-D-trehalose), most preferred is sucrose. Trehalose is available as trehalose dihydrate. In any aspect or embodiment described herein, the at least one saccharide(s) can be present in the formulation in an amount of about 10 to about 500 mM, preferably in an amount of about 200 to about 300 mM, more preferably in an amount of about 220 to about 250 mM, particularly an amount of about 220 mM or about 240 mM, most preferably in an amount of about 220 mM.

The term “amino acid” as used herein as a stabilizer refers to a pharmaceutically acceptable organic molecule possessing an amino moiety located at a-position to a carboxylic group. In any aspect or embodiment described herein, the amino acid is one or more of arginine, glycine, ornithine, lysine, histidine, glutamic acid, asparagic acid, isoleucine, leucine, alanine, phenylalanine, tyrosine, tryptophane, methionine, serine, and proline. In any aspect or embodiment described herein, the amino acid employed is the L-form. Basic amino acids, such as arginine, histidine, or lysine, are preferably employed in the form of their inorganic salts (advantageously in the form of the hydrochloric acid salts, i.e. as amino acid hydrochlorides). In any aspect or embodiment descried herein, the amino acid is methionine. In any aspect or embodiment described herein, the amino acid (such as methionine) is used at a concentration of about 5 to about 25 mM or about 10 mM.

In any aspect or embodiment described herein, the stabilizer includes or is one or more lyoprotectant. The term “lyoprotectant” as used herein refers to a pharmaceutically acceptable excipients, which protect the labile active ingredient (e.g. a protein) against destabilizing conditions during the lyophilisation process, subsequent storage and reconstitution. In any aspect or embodiment described herein, the lyoprotectants comprise, but are not limited to, the group consisting of saccharides, polyols (such as e.g. sugar alcohols), and amino acids. In any aspect or embodiment described herein, the one or more lyoprotectant is selected from the group consisting of saccharides such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine, or mixtures thereof. In any aspect or embodiment described herein, the one or more lyoprotectant is used in an amount of about 10 to 500 mM, preferably in an amount of about 10 to about 300 mM and more preferably in an amount of about 100 to about 300 mM.

In any aspect or embodiment described herein, the stabilizer includes or is one or more antioxidant. The term “antioxidant” as used herein refers to a pharmaceutically acceptable excipient, which prevent oxidation of the active pharmaceutical ingredient. In any aspect or embodiment described herein, the one or more antioxidant comprises, but are not limited to, ascorbic acid, gluthathione, cysteine, methionine, citric acid, and EDTA. In any aspect or embodiment described herein, the one or more antioxidant is used in an amount of about 0.01 to about 100 mM, preferably in an amount of about 5 to about 50 mM and more preferably in an amount of about 5 to about 25 mM.

In any aspect or embodiment described herein, the compositions or formulations described herein further comprise one or more tonicity agents. The term “tonicity agents” as used herein refers to pharmaceutically acceptable excipients used to modulate the tonicity of the formulation. The formulation can be hypotonic, isotonic or hypertonic. Isotonicity in general relates to the osmotic pressure of a solution, usually relative to that of human blood serum (around 250-350 mOsmol/kg). The formulation according to the present disclosure can be hypotonic, isotonic or hypertonic, but will preferably be isotonic. In any aspect or embodiment described herein, an isotonic formulation is liquid or liquid reconstituted from a solid form, e.g. from a lyophilized form, and denotes a solution having the same tonicity as some other solution with which it is compared, such as physiologic salt solution and the blood serum. In any aspect or embodiment described herein, the one or more tonicity agents includes or is selected from sodium chloride, potassium chloride, glycerine and any component from the group of amino acids or sugars, in particular glucose. In any aspect or embodiment described herein, the one or more tonicity agents is used in an amount of about 5 mM to about 500 mM. Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is trehalose.

The term “polyols” as used herein denotes pharmaceutically acceptable alcohols with more than one hydroxy group. In any aspect or embodiment described herein, the one or more polyols is selected from mannitol, sorbitol, glycerine, dextran, glycerol, arabitol, propylene glycol, polyethylene glycol, and combinations thereof. In any aspect or embodiment described herein, the one or more polyols is used in an amount of about 10 mM to about 500 mM, particularly in an amount of about 10 to about 250 mM and more particularly in an amount of about 200 to about 250 mM.

In any aspect or embodiment described herein, the composition or formulations described herein may further includes adjuvants (such as preservatives, wetting agents, emulsifying agents and dispersing agents). Prevention of presence of microorganisms may be ensured both by sterilization procedures, and by the inclusion of various antibacterial and antifungal agents, e.g. paraben, chlorobutanol, phenol, sorbic acid, and the like. In any aspect or embodiment described herein, the one or more preservative is used in an amount of about 0.001 to about 2% (w/v). In any aspect or embodiment described herein, the one or more preservative is selected from ethanol, benzyl alcohol, phenol, m-cresol, p-chlor-m-cresol, methyl or propyl parabens, benzalkonium chloride.

Advantageously, the present disclosure relates to a pharmaceutical composition or formulation as defined above, which is in the form of a liposome, or nanoparticles, or in the form of a solution. An advantageous solution is a solution comprising from 1 to 15%, in particular about 10% of mannitol. The solution should be iso-osmolar.

### Definitions

Unless otherwise defined, all technical and scientific terms used herein have the same meaning as commonly understood by one of ordinary skill in the art to which this disclosure belongs. The terminology used in the description is for describing particular embodiments only and is not intended to be limiting of the present disclosure.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure. The upper and lower limits of these smaller ranges which may independently be included in the smaller ranges is also encompassed within the present disclosure, subject to any specifically excluded limit in the stated range. Where the stated range includes one or both of the limits, ranges excluding either both of those included limits are also included in the present disclosure.

As used herein, the following terms may have meanings ascribed to them below, unless specified otherwise. However, it should be understood that other meanings that are known or understood by those having ordinary skill in the art to which the present disclosure belongs are also possible, and within the scope of the present disclosure.

It must be noted that as used herein and in the appended claims, the singular forms “a”, “and”, and “the” include plural references (i.e., refer to one or to more than one or at least one) to the grammatical object of the article. By way of example, “an element” means one element or more than one element.

The term “about” as it is used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “affinity reagent” is any molecule that specifically binds to a target molecule, for example, to identify, track, capture or influence the activity of the target molecule. The affinity reagent identified or recovered by the methods described herein are “genetically encoded,” for example an antibody, peptide or nucleic acid, and are thus capable of being sequenced. The terms “protein,” “polypeptide,” and “peptide” are used interchangeably herein to refer to two or more amino acids linked together.

The term “molecular display system” is any system capable of presenting a library of potential affinity reagents to screen for potential binders to a target molecule/protein or ligand. Examples of molecular display systems include phage display, bacterial display, yeast display, ribosome display and mRNA display. In any aspect or embodiment described herein, phage display is used.

The term “antibody” as used herein in the specification and claims includes whole antibodies and any antigen binding fragment (e.g., “antigen-binding portion”) or single chains thereof. For example, in any aspect or embodiment described herein “antibody” as used herein in the specification and claims refers to a protein or immunoglobulin comprising at least two heavy chains (H chains) and two light chains (L chains) connected or stabilized by disulfide bonds. Each heavy chain is comprised of a heavy chain variable region (VH) and a heavy chain constant region (C_{H}). Each light chain is comprised of a light chain variable region (V_{L}) and a light chain constant region (C_{L}). The heavy chain-constant region comprises three heavy chain-constant domains (C_{H1}, C_{H2}, and C_{H3}) or four heavy chain-constant domains (IgM-type or IgE-type antibodies; C_{H1}, C_{H2}, C_{H3} and C_{H4}) wherein the first constant domain C_{H1} is adjacent to the variable region and may be connected to the second constant domain C_{H2} by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs (CDR1, CDR2, and CDR3) and four FRs (FR1, FR2, FR3, and FR4), arranged from amino-terminus to carboxy-terminus in the following order: FR1, CDR1, FR2, CDR2, FR3, CDR3, FR4. The constant regions of the antibodies can mediate the binding of the immunoglobulin to host tissues or factors, including various cells of the immune system (e.g., effector cells) and the first component (C1q) of the classical complement system. The variable regions of the heavy and light chains contain a binding domain that interacts with an antigen. The heavy chain constant regions may be of any type, such as γ-, δ-, α-, μ.- or ε-type heavy chains. In any aspect or embodiment described herein, the heavy chain constant region of the antibody is a γ-chain. Furthermore, the light chain constant region may also be of any type, such as κ-type light chain or λ-type light chain. In any aspect or embodiment, the light chain constant region of the antibody is a κ-chain.

The terms “γ- (δ-, β-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. In particular, the amino acid sequence of the constant domains of a γ-type (especially γ1-type) heavy chain is at least 95%, especially at least 98%, identical to the amino acid sequence of the constant domains of a human γ (especially the human γ1) antibody heavy chain. Furthermore, the amino acid sequence of the constant domain of a κ-type light chain is in particular at least 95%, especially at least 98%, identical to the amino acid sequence of the constant domain of the human K antibody light chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody.

The term “isotype” as used herein in the specification and claims refers to the antibody class (e.g., IgG, IgD, IgA, IgM, or IgE) that is encoded by the heavy chain constant region genes (γ-, δ-, α-, μ- or ε-heavy chain constant genes, respectively).

The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The antigen-binding portion of an antibody as used herein in the specification and claims usually refers to full length or one or more fragments (e.g., antigen binding fragment) of an antibody that retains the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and C_{H1} domains; a F(ab)₂ fragment, a bivalent fragment comprising two Fab fragments, each of which binds to the same antigen, linked by a disulfide bridge at the hinge region; a Fd fragment consisting of the V_{H} and C_{H1} domains; a Fv fragment consisting of the V_{L} and V_{H} domains of a single arm of an antibody; and a dAb fragment, which consists of a V_{H} domain.

The term “Fab part” as used herein in the specification and claims refers to a part of the antibody comprising the heavy and light chain variable regions (V_{H} and V_{L}) and the first domains of the heavy and light chain constant regions (C_{H1} and C_{L}). In cases where the antibody does not comprise all of these regions, then the term “Fab part” only refers to those of the regions V_{H}, V_{L}, C_{H1} and C_{L} which are present in the antibody. In certain embodiments, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. For example, the Fab part comprises at least the V_{H} region of the antibody.

The term “Fc part” as used herein in the specification and the claims is a part of the antibody comprising the heavy chain constant regions 2, 3 and—where applicable—4 (C_{H2}, C_{H3} and C_{H4}). In particular, the Fc part comprises two of each of these regions. In cases where the antibody does not comprise all of these regions, then the term “Fc part” only refers to those of the regions OH₂, H₃ and C_{H4} which are present in the antibody. Preferably, the Fc part comprises at least the C_{H2} region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

The term “antibody” as used herein in the specification and claims, refer in certain embodiments to a population of antibodies of the same kind. In particular, all antibodies of the population exhibit the features used for defining the antibody. In any aspect or embodiment described herein, all antibodies in the population have the same amino acid sequence.

The term “antibody” as used herein in the specification and claims also includes fragments and derivatives of said antibody. A “fragment or derivative” of an antibody as used herein in the specification and claims is a protein or glycoprotein which is derived from said antibody and is capable of binding to the same antigen, in particular to the same epitope as the antibody. Thus, a fragment or derivative of an antibody herein generally refers to a functional fragment or derivative. In any aspect or embodiment described herein, the fragment or derivative of an antibody comprises a heavy chain variable region. It has been shown that the antigen-binding function of an antibody can be performed by fragments of a full-length antibody or derivatives thereof. Examples of fragments of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain C_{H1} of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. In any aspect or embodiment described herein, derivatives of an antibody include antibodies that bind to or compete with the same antigen as the parent antibody, but which have a different amino acid sequence than the parent antibody from which it is derived. These antibody fragments and derivatives are obtained using conventional techniques known to those with skill in the art, and the fragments are screened for utility in the same manner as are intact antibodies. The term “chimeric antibody” as used herein in the specification and claims is an antibody having a heavy chain variable region and a light chain variable region from one species linked to a heavy chain constant region and a light chain constant region from a different species, respectively (e.g., combining genetic material from a nonhuman source with genetic material from a human being).

The term “human antibody” as used herein in the specification and claims includes antibodies having variable regions in which both the FR and CDR regions are derived from human germline immunoglobulin sequences. Furthermore, if the antibody contains a constant region, the constant region also is derived from human germline immunoglobulin sequences. The human antibodies of the present disclosure can include amino acid residues not encoded by human germline immunoglobulin sequences (e.g., mutations introduced by random or site-specific mutagenesis in vitro or by somatic mutation in vivo). However, the term “human antibody” as used herein the specification and claims does not include antibodies in which CDR sequences derived from the germline of another mammalian species have been grafted onto human framework sequences.

The term “mouse antibody” as used herein in the specification and claims includes antibodies having variable regions in which both the FR and CDR regions are derived from mouse germline immunoglobulin sequences. Furthermore, if the antibody contains a constant region, the constant region also is derived from mouse germline immunoglobulin sequences. The mouse antibodies of the present disclosure can include amino acid residues not encoded by mouse germline immunoglobulin sequences (e.g., mutations introduced by random or site-specific mutagenesis in vitro or by somatic mutation in vivo). However, the term “mouse antibody” as used herein in the specification and claims does not include antibodies in which CDR sequences derived from the germline of another mammalian species have been grafted onto mouse framework sequences.

The term “rabbit antibody” as used herein in the specification and claims includes antibodies having variable regions in which both the FR and CDR regions are derived from mouse germline immunoglobulin sequences. Furthermore, if the antibody contains a constant region, the constant region also is derived from mouse germline immunoglobulin sequences. The mouse antibodies of the invention can include amino acid residues not encoded by mouse germline immunoglobulin sequences (e.g., mutations introduced by random or site-specific mutagenesis in vitro or by somatic mutation in vivo). However, the term “mouse antibody” as used in the specification and claims does not include antibodies in which CDR sequences derived from the germline of another mammalian species have been grafted onto mouse framework sequences.

The term “humanized antibody” as used herein in the specification and claims refers to an antibody from non-human species whose protein sequences have been modified to increase similarity to antibody variants produced naturally in humans, such as an amino acid sequence characteristic of an antibody derived from a non-human has replaced a corresponding position of a human antibody. Examples of the humanized antibody include an antibody having heavy chain CDR1 to CDR3 and light chain CDR1 to CDR3 derived from an antibody prepared from a non-human antibody and in which all other regions comprising respective four framework regions (FRs) of the heavy chain and the light chain are derived from a human antibody. Such an antibody may be referred to as a CDR-grafted antibody. The term “humanized antibody” may include a human chimeric antibody. A “human chimeric antibody” is an antibody based on an antibody derived from a non-human in which a constant region of the antibody derived from a non-human has been replaced with a constant region of a human antibody. For increasing the ADCC activity of the human chimeric antibody, for example, the subtype of the human antibody used for the constant region can be IgG1.

A “monoclonal antibody” is an antibody produced by a single clone of B lymphocytes from mouse or rabbit or by a cell, e.g., HEK293 cell, into which the light and heavy chain genes of a single antibody have been transfected. Monoclonal antibodies are produced by methods known to those of ordinary skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

The term “human monoclonal antibody” as used herein in the specification an claims refers to a monoclonal antibody that has variable regions in which both the FR and CDR regions are derived from human germline immunoglobulin sequences.

The term “isolated antibody” as used herein the specification and claims refers to an antibody that is substantially free of other antibodies having different antigenic specificities (e.g., an isolated bispecific antibody as described herein that specifically binds the protein of interest). Moreover, an isolated antibody can be substantially free of other cellular material and/or chemicals.

The term “epitope” includes any protein determinant capable of specific binding to an immunoglobulin, or fragment. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

The term “functional epitope” means the residues within the epitope that make energetic contributions to the binding interaction and/or involved in any physiological or biochemical function of the protein.

A target amino acid sequence is “derived” from or “corresponds” to a reference amino acid sequence if the target amino acid sequence shares a homology or identity over its entire length with a corresponding part of the reference amino acid sequence of at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In any aspect or embodiment described herein, a target amino acid sequence which is “derived” from or “corresponds” to a reference amino acid sequence is 100% homologous, or in particular 100% identical, over its entire length with a corresponding part of the reference amino acid sequence. The “homology” or “identity” of an amino acid sequence or nucleotide sequence is preferably determined according to the present disclosure over the entire length of the reference sequence or over the entire length of the corresponding part of the reference sequence that corresponds to the sequence that homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% homologous or identical, especially identical, to the respective amino acid sequences of the parent antibody. In any aspect or embodiment described herein, the antibody derived from (i.e. derivative of) a parent antibody comprises the same CDR sequences as the parent antibody, but differs in the remaining sequences of the variable regions.

By “homology” is meant two or more nucleic acid or amino acid sequences is partially or completely identical. In any aspect or embodiment described herein, the homologous nucleic acid or amino acid sequence has 30%, 40%, 50%, 60%, 70%, 80%, 90%, or 95% sequence similarity or identity to an nucleic acid encoding the reference nucleic acid or amino acid sequence.

“Homologs” can be naturally occurring or created by artificial synthesis of one or more nucleic acids or polypeptides having related sequences, or by modification of one or more nucleic acid or amino acid to produce related nucleic acid or amino acid sequences. If the homology between two nucleic acid or amino acid sequences is not expressly described, homology can be inferred by a nucleic acid or amino acid comparison between two or more sequences. If the sequences demonstrate some degree of sequence similarity, for example, greater than about 30% at the primary amino acid structure level, it is concluded that they share a common ancestor. For purposes of the present disclosure, genes are homologous if the nucleic acid or amino acid sequences are sufficiently similar to allow recombination and/or hybridization under low stringency conditions. In addition, polypeptides are regarded as homologous if their nucleic acid sequences are sufficiently similar to allow recombination or hybridization under low stringency conditions.

The term “specific binding” as used in the specification and claims preferably means that an agent such as an antibody, binds stronger to a target, such as an epitope for which it is specific, compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold, or 1000-fold lower than the dissociation constant for the target to which the agent does not bind specifically. Furthermore, the term “specific binding” in particular indicates a binding affinity between the binding partners with an affinity constant Kₐ of at least 10⁶ M⁻¹, preferably at least 10⁷ M⁻¹, more preferably at least 10⁸ M⁻¹. An antibody specific for a certain antigen in particular refers to an antibody that is capable of binding to said antigen with an affinity having a Kₐ of at least 10⁶ M⁻¹, preferably at least 10⁷ M⁻¹, more preferably at least 10⁸ M⁻¹.

The present disclosure provides, in any aspect or embodiment described herein, an antibody that binds to a protein of interest, wherein a dissociation rate constant K_{d} thereof is not less than a first specified value and an association rate constant Kₐ thereof is not less than a second specified value. The first specified value may be 1×10⁻⁴ s⁻¹ or more, 2×10⁻⁴ s⁻¹ or more, 3×10⁻⁴ s⁻¹ or more, 4×10⁻⁴ s⁻¹ or more, 5×10⁻⁴ s⁻¹ or more, 6×10⁻⁴ s⁻¹ or more, 7×10⁻⁴ s⁻¹ or more, or 8×10⁻⁴ s⁻¹ or more. The second specified value may be 1×10⁻⁴ M⁻¹ s⁻¹ or more, 1.5×10⁻⁴ M⁻¹s⁻¹ or more, or 2×10⁻⁴ M⁻¹ s⁻¹ or more. K_{D} of the antibody may be 1 nM to 100 nM, 10 nM to 50 nM, 20 nM to 40 nM, or 30 nM to 40 nM.

Combinations of the first specified value and the second specified value are as follows: the second specified value may be 1×10⁻⁴ or more and the first specified value may be 1.times.10⁻⁴ s⁻¹ or more; the second specified value may be 1.5×10⁻⁴ M⁻¹ s⁻¹ or more and the first specified value may be 2×10⁻⁴ s⁻¹ or more; or the second specified value may be 2×10⁻⁴ M⁻¹ s⁻¹ or more and the first specified value may be 3×10⁻⁴ s⁻¹ or more. The upper limit of Kₐ and K_{d} may be within the range of Kₐ and K_{d} of antibodies obtained as described herein.

The present disclosure provides an antibody that binds to a target protein or protein of interest, wherein a dissociation rate constant K_{d} thereof is 5×10⁻⁴ s⁻¹ or more and an association rate constant Kₐ thereof is 1×10⁻⁴ M⁻¹s⁻¹ or more. The dissociation rate constant K_{d} may be, for example, 1×10⁻⁴ s⁻¹ or more, 2×10⁻⁴ s⁻¹ or more, 3×10⁻⁴ s⁻¹ or more, 4×10⁻⁴ s⁻¹ or more, 5×10⁻⁴ s⁻¹ or more, 6×10⁻⁴ s⁻¹ or more, 7×10⁻⁴ s⁻¹ or more, or 8×10⁻⁴ s⁻¹ or more. The association rate constant Kₐ may be 1×10⁻⁴ M⁻¹s⁻¹ or more, 1.5×10⁻⁴ M⁻¹s⁻¹ or more, or 2×10⁻⁴ M⁻¹s⁻¹ or more. K_{D} may be 1 nM to 100 nM, 10 nM to 50 nM, 20 nM to 40 nM, or 30 nM to 40 nM. Particularly, combinations of Kₐ and K_{d} are as follows: Ka may be 1×10⁻⁴ M-s⁻¹ or more and K_{d} may be 1×10⁻⁴ s⁻¹ or more; Kₐ may be 1.5×10⁻⁴ M⁻¹s⁻¹ or more and Kₐ may be 2×10⁻⁴ s⁻¹ or more; or Kₐ may be 2×10⁻⁴ M⁻¹s⁻¹ or more and K_{d} may be 3×10⁻⁴ s⁻¹ or more. The antibody having the association rate constant and the dissociation rate constant binds to the protein of interest rapidly and also dissociates from the protein of interest rapidly. The upper limit of Kₐ and K_{d} may be within the range of Kₐ and K_{d} of antibodies obtained by immunizing an animal.

Each of the association rate constant Kₐ and the dissociation rate constant K_{d} of an antibody can be determined, for example, by surface plasmon resonance (SPR) measurement. SPR measurement for binding between an antibody and an antigen is well known and those skilled in the art will be able to calculate the association rate constant Kₐ and the dissociation rate constant K_{d} of the antibody based on a well-known technique. In SPR measurement, the association rate constant can be calculated from variation of RU in a phase in which an analyte is flowed at a fixed concentration (association phase) and then, the dissociation constant can be calculated from variation of RU in a phase in which running buffer is flowed (dissociation phase). Measurement can be performed by using single-cycle kinetics. Analysis can be performed by bivalent analysis. Curve fitting of an approximate curve to a measured SPR sensorgram can be performed by using a kinetic titration 1:1 interaction model. For details of curve fitting, one can see Karlsson, R., Katsamba, P. S., Nordin, H., Pol, E. and Myszka, D. G. (2006). “Analyzing a kinetic titration series using affinity biosensors.” Anal. Biochem. 349 (1): 136-47. Additionally, assessment of the association rate constant Kₐ and the dissociation rate constant K_{d} of an antibody can be performed using surface-based (heterogeneous) methods including SPR, biolayer interferometry (BLI) and enzyme linked immunosorbent assays (ELISA). Schuck 1997; Gauglitz 2008; Friguet et al. 1985.

The association rate constant Kₐ and the dissociation rate constant K_{d} of an antibody can also be determined, for example, by using an SPR instrument, such as Biacore™ commercially available from GE Healthcare, according to the manufacturer's manual. For example, the SPR measurement instrument also includes a program for determining Kₐ and K_{d} and can calculate Kₐ and K_{d} from an SPR sensorgram. For example, an SPR sensorgram obtained by a Biacore™ instrument can be subjected to analysis in which Biacore T200 evaluation software is used and a bivalent analyte model is adopted as a fitting model, thereby deriving a fitting curve, from which Kₐ, K_{d}, and KD as kinetics parameters of an antibody or an ADC can be calculated.

In any aspect or embodiment described herein, a competitive assay can be used to test whether antibodies have binding properties similar to each other. An antibody that competes with a certain antibody for binding to an antigen thereof can be identified for example by a competitive assay well known to those skilled in the art. When an antibody can block binding of a desired antibody to an antigen thereof, for example, by at least 20%, preferably at least 20 to 50%, further preferably at least 50%, more preferably 60%, more preferably 70%, more preferably 80%, and especially preferably 90% or more, in the competitive assay, the antibody can be identified as an antibody that competes for binding to the same antigen. In any aspect or embodiment described herein, a competitive antibody can be identified by a cross-blocking assay or a competitive ELISA assay. In the cross-blocking assay, an antigen is coated onto, for example, a microtiter plate, and a competitive antibody entity as a candidate is added thereto and incubated to allow binding between the antigen and the candidate antibody to form. Subsequently, the desired antibody is labelled, then added additionally to the well, incubated, and washed. One can determine whether the candidate antibody competed or not by quantifying the amount of the desired antibody that is bound. When competition exists, the amount of the label remaining in the well should be decreased.

Generally, in the competitive assay, the fact that a first antibody causes dissociation of binding of a second antibody to an antigen does not always mean that the second antibody causes dissociation of binding of the first antibody to the antigen. This can be easily understood by imagining a case where the first antibody shows extremely strong binding to the antigen compared to the second antibody. Identification of an antibody having a similar binding property may be achieved by confirming that the first antibody causes dissociation of binding of the second antibody to an antigen and the second antibody causes dissociation of binding of the first antibody to the antigen. Herein, such a competitive state is referred to as “the first antibody and the second antibody mutually compete with each other for binding to an antigen.”

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In any aspect or embodiment described herein, one or more of the present binders/compounds described herein, are coadministered in combination with at least one additional bioactive agent, especially including an anticancer agent. In any aspect or embodiment described herein, the co-administration of compounds results in synergistic activity and/or therapy, including anticancer activity.

The term “effective amount/dose,” “pharmaceutically effective amount/dose,” “pharmaceutically effective amount/dose,” or “therapeutically effective amount/dose” can mean, but is in no way limited to, that amount/dose of the active pharmaceutical ingredient sufficient to prevent, inhibit the occurrence, ameliorate, delay or treat (alleviate a symptom to some extent, preferably all) the symptoms of a condition, disorder or disease state. The effective amount depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the agent. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present disclosure, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The term “pharmacological composition,” “therapeutic composition,” “therapeutic formulation,” or “pharmaceutically acceptable formulation” can mean, but is in no way limited to, a composition or formulation that allows for the effective distribution of an agent provided by the present disclosure, which is in a form suitable for administration to the physical location most suitable for their desired activity, e.g., systemic administration.

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “systemic administration” refers to a route of administration that is, e.g., enteral or parenteral, and results in the systemic distribution of an agent leading to systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect. Administration routes which lead to systemic absorption include, without limitations: intravenous, subcutaneous, intraperitoneal, inhalation, oral, intrapulmonary, and intramuscular. The rate of entry of a drug into the circulation has been shown to be a function of molecular weight or size. The use of a liposome or other drug carrier comprising the compounds of the present disclosure can potentially localize the drug, for example, in certain tissue types, such as the tissues of the reticular endothelial system (RES). A liposome formulation which can facilitate the association of drug with the surface of cells, such as, lymphocytes and macrophages is also useful.

The term “conservative substitution” or “conservative mutation” as used herein in the specification and claims refers to replacement of an amino acid by an amino acid of similar structure (such as size) and characteristics or chemical nature, such as where a hydrophobic amino acid is replaced by another hydrophobic amino acid (e.g., replacing a leucine with an isoleucine). In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitution” or “conservative mutation”. In any aspect or embodiment described herein the term “conservative mutations” or “conservative substitutions” can refer to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “therapeutically effective amount or dose” includes a dose of a drug that is capable of achieving a therapeutic effect in a subject in need thereof. For example, a therapeutically effective amount of a drug can be the amount that is capable of preventing or relieving one or more symptoms associated with a disease or disorder, e.g., tissue injury or muscle-related disease or disorder. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A kit is any manufacture (e.g. a package or container) comprising at least one reagent, e.g. a probe, for specifically detecting a marker of the present disclosure. The manufacture may be promoted, distributed, or sold as a unit for performing the methods of the present disclosure. The reagents included in such a kit comprise antibodies for use in treating a disease and/or disorder and/or detecting/binding the protein of interest. In addition, the kits of the present disclosure may preferably contain instructions which describe the therapeutic use of the antibodies and/or a suitable detection assay utilizing the antibodies. Such kits can be conveniently used, e.g., in clinical settings, to diagnose or treating patients exhibiting symptoms of a disease and/or disorder.

### OTHER EMBODIMENTS

The preceding detailed description of the present disclosure is provided to aid those skilled in the art in practicing the present disclosure. Those of ordinary skill in the art may make modifications and variations in the embodiments described herein without departing from the spirit or scope of the present disclosure. Although any methods and materials similar or equivalent to those described herein can also be used in the practice or testing of the present disclosure, the preferred methods and materials are now described. Unless otherwise defined, all technical and scientific terms used herein have the same meaning as commonly understood by one of ordinary skill in the art to which this disclosure belongs. The terminology used in the description of the present disclosure herein is for describing particular embodiments only and is not intended to be limiting of the present disclosure. All publications, patent applications, patents, figures and other references mentioned herein are expressly incorporated by reference in their entirety.

### EXAMPLES

### Example 1: Cloning and Expression and Screening

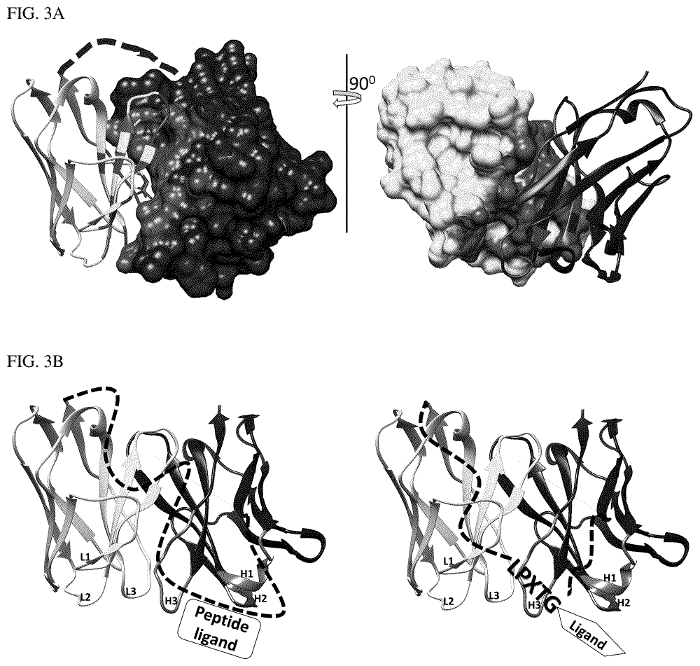

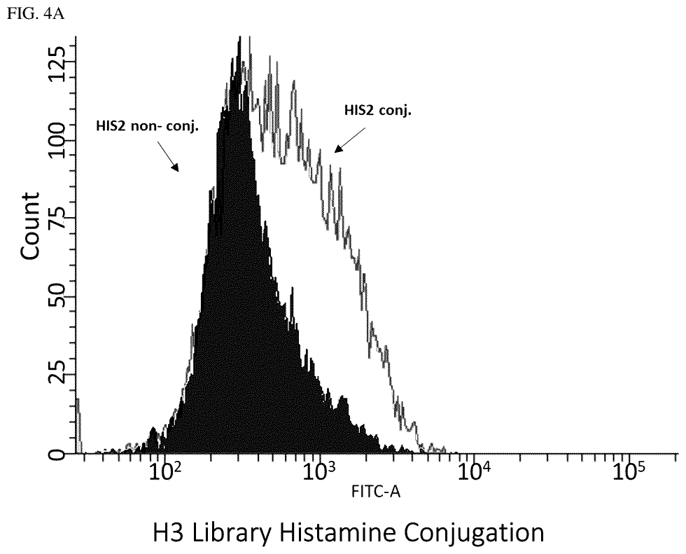

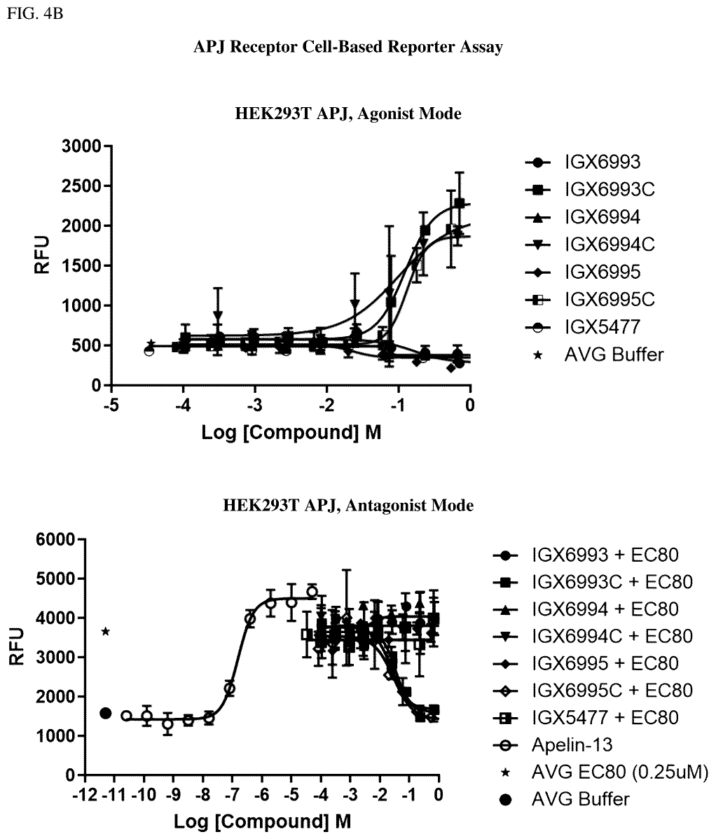

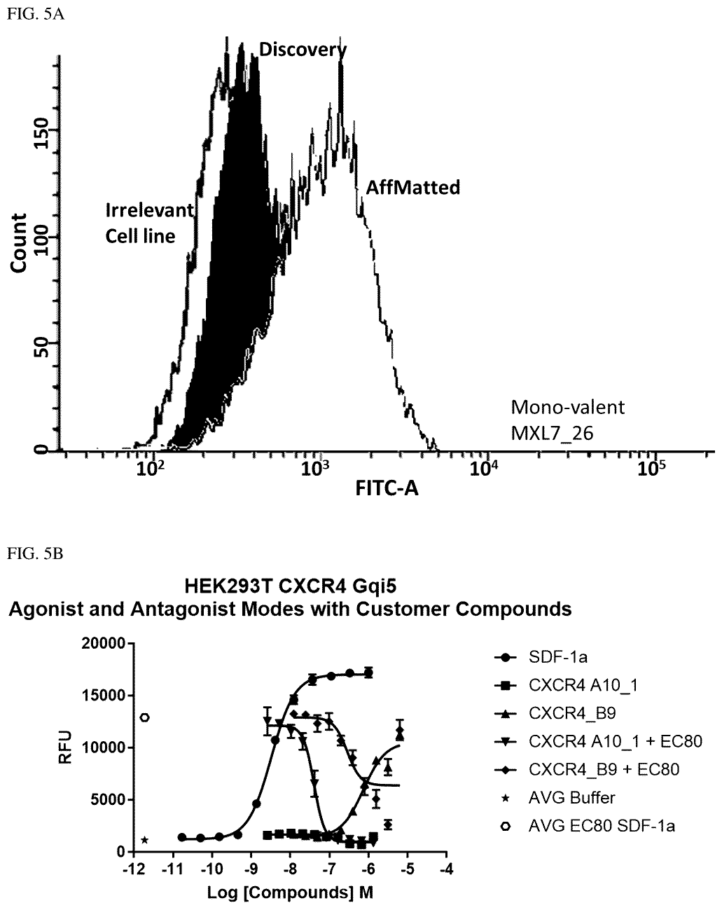

Sortase enzyme purchased from Active Motif (Carlsbad, CA) was used to conjugate the apelin17 ligand (KFRRQRPRLSHKGPMPF) to the polyclonal hyperphage (in naïve discovery mode) or monovalent phage (in AffMat mode) libraries before screening. There are five copies of the scFv on each hyperphage in the discovery library. As such, even an inefficient sortase reaction (ca. 40% efficiency) probably adds at least one ligand molecule to at least one of the five gpIII fusion scFvs. The conditions for sortase conjugation of a few peptides and small molecules into a polyclonal phage library have been determined, including the Histamine receptor 2 (small molecule, data not shown), and APJ17 (the 17 amino acid apelin peptide) (FIG. 3). Free ligand after conjugation is removed by PEG precipitation of the library. With small molecules, overnight dialysis is also used. With the power of using sortase fusion a single IgG clone can be screened both with and without ligands. This is not something that can be readily done with genetic fusions, since removal of a ligand in genetic fusions would drastically alter the CDR structure, and by default the IgG structure. In a preliminary study on the APJ screen, although both conjugated and non-conjugated IgGs bound to the OE APJ cell lines, only the ligand-conjugated clone showed functionality in a cell-based reporter assay. (FIG. 3B).

### Experimental Design

(1) Targets. The DLA2.0 discovery screening focuses on sortase conjugation of small molecules. Specifically, a subset of 4 of the 6 GPCRs from Table 2. (2) Library construction, preparation of sortase-conjugated libraries and whole cell screening. scFvs are initially used in naïve screens and converted to IgGs for final validation. (3) Ligand selection. Ligands that are commercially available or that can be readily made by a simple chemical modification of an existing ligand are readily adapted for use in the current processes. Many such ligands are available. (4) Construction of Clustered Regularly Interspaced Short Palindromic Repeats (CRISPR) cell lines for producing OE receptor and reporter cell lines. Availability of an appropriate cell line is often rate-limiting. The target cell line is generated in house using a CRISPR process. To date we have made 5 GPCR cell lines and validated with flow cytometry (80% success rate). The appropriate cells can be purchased from Multispan. Cell lines having at least 200,000 cell-surface receptors will initially be used. Our in-house CRISPR-constructed cell lines will be quality controlled for a similar number of surface receptors.

### Example 2: Characterization of Cloned and Expressed IgGs in Cell Line Reporter Assays and Applications

mAbs that bind to the native structure and are functional will be considered for further characterization. They will be validated for applications such as immunohistochemistry (IHC), immunocytochemistry (ICC), and flow cytometry, as well as for functionality in cell-based reporter assays. Only the latter requires function to pass validation. But the others do require high affinity and specificity.

TABLE 1
G Protein-Coupled Receptors (GPCRs) and
Ligands used for Directed Ligand Binding

| GPCR | Disease | Ligand | Results |
|---|---|---|---|
| CXCR4 | HIV infection, various | Peptide | Antagonist, AffMatted |
| | types of cancer, breast | | by mutating ligand |
| | cancer | | shows partial agonist |
| NTSR1 | Low blood pressure, | Peptide | Agonist |
| | inflammatory bowel | | |
| | disease, cancer | | |
| NTSR2 | Cancer | Peptide | Antagonist |
| CCR5 | HIV infection, various | Chemokine | Partial agonist |
| | types of cancer, | protein | |
| | autoimmune disease, | | |
| | stroke | | |

### Experimental Design.

(1) Evaluate binders in functional IgG assays, flow cytometry, and immunofluorescence. The initial discovery and affinity matured scFv binders from both genetic and enzymatic conjugations will be converted to purified IgG protein and tested in research applications (as specific examples: flow cytometry, immunohistochemistry, and/or ELISA), as well as for functional activity (primarily live-cell reporter assays). Characterizations will examined with both OE cell lines and (to be useful in practical applications) with cell lines expressing what would be considered a wild-type amount of the receptor (see Research Use Only section below). When available, CRISPR knock-out cell lines will also be used as a negative control. IgG clones that do not bind with high enough affinity will be affinity matured using phage display. We assume final clones should have K_{D}'s in the low nM. (2) Binding kinetics, functional studies and determining binding specificity of mAbs. kinetic measurements: The binding kinetics of IgGs will be determined using either titration ELISA against whole cells, bio-layer interferometry (BLI, Octet), or surface plasmon resonance (SPR, Biacore). (3) Making Research Use Only, diagnostic mAbs, and developing a commercial pipeline. Research use only mAbs can be quite different in their specifications than mAbs used in therapeutics. The proof-of-concept experiments have shown success in validating our GPCR mAbs in flow cytometry, including FACS and live-cell functional assays but these mAbs could have added value in diagnostic and research applications. To be useful for these functions, the clones may be affinity matured and tested for specificity against panels of (related) GPCRs. Subtracting an affinity mature library against the related cell-line first will be used. These applications will not require function beyond binding in a specific assay since function is not always a required specification of a research use only mAb. (4) Functional studies. The IgGs will be outsourced for functional studies. Unless, because of a lack of third-party availability, they are required to be made in house. (5) Specificity: In the supplier's test system (Multispan), binding of a mAb to a tagged test GPCR receptor panel is measured by flow cytometric analysis using Geometric Mean of fluorescence intensity. The Relative Binding Index (RBI) is determined by normalizing the Geometric Mean of a test mAb binding to that of an anti-tag mAb binding. RBI for the test mAb to the target GPCR is set at 100%. More than 200 GPCRs are available, between 10 and 20 relevant (i.e., related) GPCRs will be chosen. Members of most of the A-F GPCR classification will be used, prioritized on availability of both (i) appropriate OE cell lines and (ii) live-cell reporter assays.

### Example 3: Potential for Phase II Studies

Extend the method to other cell-surface receptor-types: ion channels and enzymes. To date, most ion channel drug development has focused on identifying and developing small molecule and peptide modulators. Many ion channel modulators have been discovered from studies of naturally occurring substances, such as toxins from plants and venomous animals The conotoxin family is the most well-known example of the animal derived toxins with ziconotide, a selective Cav2.2 antagonist, as a frequently cited example of a synthetic peptide analogue of cone snail ω-conotoxin used for the treatment of severe chronic pain.

### Experimental Design

(1) Potential ion channel targets with ligands having the appropriate chemical linker modifications conducive to the methods of the present disclosure are listed in Table 2. The final choice of targets will be triaged as was done for GPCRs: a selection will be made based on the availability of an appropriate cell line expressing up to 1 million receptors on the cell surface, a linker-modified ligand, and availability of third parties to perform the patch-clamp assays. (2) Characterization of ion channel mAbs will follow the path as that described for the GPCR-derived DLA2.0 mAbs. See sections above for biochemical and biophysical characterizations. (3) Evaluate binders in functional IgG assays, flow cytometry, and immunofluorescence. The initial discovery and AffMatted scFv binders from enzymatic conjugations will be converted to purified IgG protein and tested in research applications (as specific examples: flow cytometry, immunohistochemistry, and/or ELISA), as well as for functional activity (primarily patch clamp assays). Characterizations will made with both OE cell lines and (to be useful in practical applications) with cell lines expressing what would be considered a wild-type amount of the receptor (see research use only section below). (4) Binding kinetics, functional studies and determining binding specificity of mAbs. The binding kinetics of IgGs derived from the methods of the present disclosure will be determined using either titration ELISA against whole cells, bio-layer interferometry (BLI, Octet), or surface plasmon resonance (SPR, Biacore). (5) Making Research Use Only, diagnostic mAbs and developing a commercial pipeline will be performed as discussed above for GPCRs. (6) Functional studies. The IgGs will be outsourced for functional studies (as examples: Fluxion, Sophion, Reaction Biology).

TABLE 2
GPCR ligands covalently conjugated to the C-terminus of “GGGGS” peptide ready for sortase conjugation.

| | | GPCR | | | |
|---|---|---|---|---|---|
| GPCR Target | GPCR | Structure | Ligand | Ligand | Ligand Chemical Compound |
| Name | Class | PDB ID | Type | Name | Structure |
| Histamine H2 Receptor (H2); Uniprot: P25021 | Class A | 3RZE (H1) | Orthosteric | Histamine | 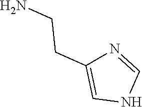 |
| 5HT-2A Receptor; Uniprot: P28223 | Class A | 6A693 | Orthosteric | Serotonin | 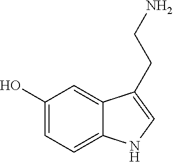 [SMILES: NCCc1c[nH]c2ccc(O)cc12] |
| Adenosine Receptor A1 (ADORA1); Uniprot: P30542 | Class A | 6D9H | Orthosteric | Adenosine | 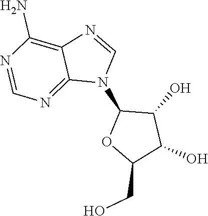 [SMILES: Nc1ncnc2c1ncn2[C@@H]1O[C@H](CO)[C@@H](O)[C@H]1O] |
| Adenosine Receptor A2a (ADORA2A); Uniprot: P29274 | Class A | 6GDG | Orthosteric | NECA (N- ethyl-5′- carboxamido adenosine) | 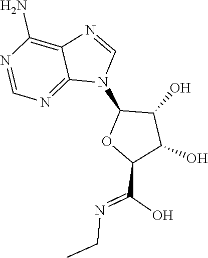 [SMILES: CC/N=C(\O)[C@H]1O[C@@H](n2cnc3c(N)ncnc32)[C@H](O)[C@@H]1O] |
| Adenosine Receptor A2a (ADORA2A); Uniprot: P29274 | Class A | 3REY | Orthosteric | XAC | 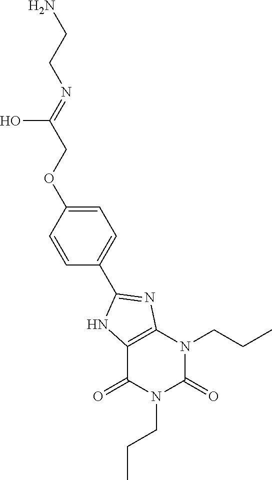 [SMILES: CCCn1c(=O)c2[nH]c(-c3ccc(OC/C(O)=N/CCN)cc3)nc2n(CCC)c1=O] |
| Smoothened homolog (SMO) Class Frizzled GPCR; Uniprot: Q99835 | Class F | 5L7D | Allosteric | Cholesterol | 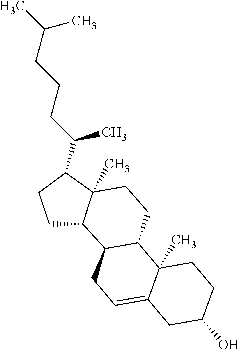 [SMILES: CC(C)CCC[C@@H](C)[C@H]1CC[C@H]2[C@@H]3CC=C4C[C@@H](O)CC[C@]4(C)[C@H]3CC[C@]12C] |

What is claimed is:

1. A method of generating an antigen binding region against a target protein, the method comprising:
(a) providing a bifunctional compound comprising an antigen binding region conjugated to a ligand that binds to a target protein;
(b) generating a first antibody library by mutating one or more amino acids in the antigen binding regions;
(c) screening the first library to identify one or more bifunctional compounds with improved binding affinity to the target protein as compared to the ligand;
(d) generating a second library by mutating the ligand of the one or more bifunctional compounds identified in step (c);
(e) screening the second library to identify one or more antigen binding regions that bind to the target protein, thereby generating an antigen binding region against the target protein.

2. The method of claim 1, wherein the ligand is a peptide.

3. The method of claim 1, wherein the ligand is a small molecule compound.

4. The method of claim 1, wherein the ligand is conjugated to the antigen binding region via a covalent bond.

5. The method of claim 1, wherein the covalent bond is a disulfide bond.

6. The method of claim 1, wherein the ligand is conjugated to the antigen binding region via a sortase reaction or a transglutaminase reaction.

7. The method of claim 1, wherein the ligand is conjugated in a complementarity-determining region (CDR) of the antigen binding region.

8. The method of claim 1, wherein the antigen binding region includes or is an antibody or an antigen binding fragment thereof.

9. The method of claim 8, wherein the ligand is conjugated to the N-terminus of a light chain variable region of the antibody, the C-terminus of a light chain variable region of the antibody, the N-terminus of a heavy chain variable region of the antibody, or the C-terminus of a heavy chain variable region of the antibody.

10. The method of claim 8, wherein the ligand is conjugated in a framework region of the variable light chain of the antibody or a variable region of the heavy chain of the antibody.

11. The method of claim 1, wherein the antigen binding region includes or is an antigen binding fragment of an antibody.

12. The method of claim 11, wherein the antigen binding fragment of the antibody is a single chain variable fragments (scFv), a fragment antigen-binding region (fab), or a Fab prime (Fab′).

13. The method of claim 12, wherein the antigen binding fragment of the antibody is a single chain variable fragment (scFv) comprising a heavy chain variable region, a light chain variable region, and a scFv peptide linker that links the heavy chain variable region and the light chain variable region, wherein the ligand is conjugated to the scFv peptide linker.

14. The method of claim 1, wherein the ligand is conjugated to the antigen binding region via a linker.

15. The method of claim 14, wherein the linker is a peptide linker or a protein linker.

16. The method of claim 15, wherein the peptide linker comprises 3-50 amino acids.

17. The method of claim 15, wherein the peptide linker comprises 3-21 amino acids.

18. The method of claim 1, wherein the first library is a phage display library or the second library is a phage display library.

19. A library comprising a plurality of bifunctional compounds, wherein each bifunctional compound comprises an antigen binding region tethered or conjugated to a ligand that binds to an epitope of a target protein.

20. The method of claim 13, wherein the scFv peptide linker is 10 to 25 amino acids.
